(19) United States
(12) Patent Application Publication
(10) Pub. No.: US 2026/0041564 A1
(43) Pub. Date: Feb. 12, 2026

(54) SPINAL REALIGNMENT AND ARTHRODESIS

(71) Applicant: 3Spine, Inc., Chattanooga, TN (US)

(72) Inventors: Christian Davis, Cambridge, MA (US); Marc M. Peterman, Duxbury, MA (US); Steven C. Humphreys, Soldotna, AK (US); Scott Hodges, Chattanooga, TN (US); Ron Yarbrough, Round Rock, TX (US); Heath Maccallum, Duxbury, MA (US); Samantha Damito, Quincy, MA (US); Miguel Monteiro, George Town, Grand Cayman (KY)

(21) Appl. No.: 19/300,513

(22) Filed: Aug. 14, 2025

Related U.S. Application Data
(63) Continuation of application No. PCT/US24/15994, filed on Feb. 15, 2024.
(60) Provisional application No. 63/445,954, filed on Feb. 15, 2023.

(51) Int. Cl. A61F 2/44; A61B 17/56; A61B 17/86; A61F 2/30

(52) U.S. Cl. CPC A61F 2/447; A61B 17/86; A61F 2/30749; A61F 2/30771; A61B 2017/564; A61F 2002/3006; A61F 2002/30182; A61F 2002/30322; A61F 2002/30622; A61F 2002/30884; A61F 2002/30891; A61F 2002/3093

(57) ABSTRACT

Disclosed are systems, methods and procedures for altering and/or realigning spinal motion segments to facilitate spinal arthrodesis procedures and patient recovery therefrom.

### CROSS-REFERENCE TO RELATED APPLICATIONS

This application is a continuation of Patent Cooperation Treaty Patent Application No. PCT/US24/15994 entitled “Spinal Realignment and Arthrodesis” filed Feb. 15, 2024, which claims the benefit of U.S. Provisional Application No. 63/445,954 entitled “Fusion Spinal Implant Systems” filed Feb. 15, 2023, the disclosures of which are each hereby incorporated by reference herein in their entireties.

### TECHNICAL FIELD

Improved fusion devices, systems and methods are disclosed, including spinal implant devices and related system components which allow a surgeon to significantly alter an alignment, spacing and/or kinematics of one or more spinal motion segments, desirably improving patient recovery and outcomes from spinal arthrodesis surgery. In many instances, altering the alignment and/or spacing of a single motion segment can dramatically improve the potential for arthrodesis to occur across the joint, as well as improve implant performance and loading, reduce complications, enhance surgical outcomes and improve patient satisfaction. In various embodiments, an implantation site can be surgically prepared and one or more system components can be at least partially inserted into the implantation site (e.g., such as an intervertebral space between adjacent vertebrae) to desirably alleviate neurological effects and/or pain, stabilize one or more vertebral segments, restore spinal stability and/or alignment, and/or improve surgical outcomes. In various embodiments, single and/or multi-level implantations and/or treatment are contemplated herein, including the use of arthrodesis implants at two or more levels of the spine and/or concurrent and/or sequential implantation of arthrodesis and motion preserving implants (e.g., joint replacement implants) at differing levels of the spine in the same surgical procedure.

### BACKGROUND OF THE INVENTION

Often the source of a patient's back pain may not be clear. Among possible causes for such pain are disease, degradation and/or injury to the vertebra and/or discs of the spine, as well as to various ancillary structures such as the lamina and/or associated facet joints. Many treatments and/or procedures have been successful in treating spinal joints to reduce pain, but sometimes performing spinal fusion surgery is warranted.

### BRIEF DESCRIPTION OF THE SEVERAL VIEWS OF THE DRAWINGS

FIGS. 1A through 1I depict various views of one exemplary embodiment of an interbody fusion implant;

FIGS. 2A through 2D depict various view of an exemplary left-side interbody device;

FIGS. 2E through 2H depict various view of an exemplary right-side interbody device;

FIGS. 21 through 2L depict various views of the left-side interbody device of FIGS. 2A through 2D and the right-side interbody device of FIGS. 2E through 2H in an exemplary implanted configuration;

FIGS. 3A and 3B depict sagittal and posterior views of a spine region having lordotic angles and transverse pedicle angles;

FIGS. 4A through 4C depicts anterior, sagittal and posterior views of a spine region with one or more fusion spinal implant systems implanted therein;

FIGS. 5A and 5B depict posterior views of a spine region with fusion spinal implants implanted therein;

FIG. 6A depicts an exemplary damaged or diseased spine segment;

FIG. 6B depicts the spine segment of FIG. 6A having altered spacing and/or angulation from two interbody implants of different sizes;

FIG. 6C depicts the spine segment of FIG. 6A having altered spacing and/or angulation from a single angled implant.

FIGS. 7A and 7B depict the spine segment of FIG. 6A with altered spacing and/or angulation from different size fusion implants of the present invention;

FIG. 8A depicts a side view of an exemplary spinal motion unit undergoing a surgical procedure in accordance with one exemplary embodiment of the present invention;

FIG. 8B depicts a side view of an exemplary spinal motion unit undergoing a surgical procedure in accordance with another exemplary embodiment of the present invention;

FIG. 8C depicts a top view of a vertebral body in which proposed resections are shown in shadow as planning boxes;

FIGS. 9A through 9C depict views of one exemplary lordotic correction obtainable through teachings of the present invention;

FIGS. 10A through 10C depict coronal views of a spine segment in a spine region with various fusion spinal implants implanted therein;

FIGS. 11A through 11C depict coronal views of a spine segment in a spine region with various fusion spinal implants implanted therein;

FIGS. 12A and 12 B depict isometric and front views of an exemplary embodiment of a fusion spinal implant component;

FIGS. 13A and 13B depict isometric and front views of another exemplary embodiment of a fusion spinal implant component;

FIG. 14 depicts a side view of another exemplary embodiment of a fusion spinal implant component;

FIGS. 15A through 15J depicts various views of another exemplary embodiment of a fusion spinal implant component;

FIGS. 16A through 16J depicts various views of another exemplary embodiment of a fusion spinal implant component;

FIGS. 17A through 17C depict various views of another exemplary embodiment of a fusion spinal implant component;

FIGS. 18A through 18H depict various views of another exemplary embodiment of a fusion spinal implant component;

FIGS. 19A through 19I depict various views of another exemplary embodiment of a fusion spinal implant component;

FIGS. 20A through 20I depict various views of another exemplary embodiment of a fusion spinal implant component

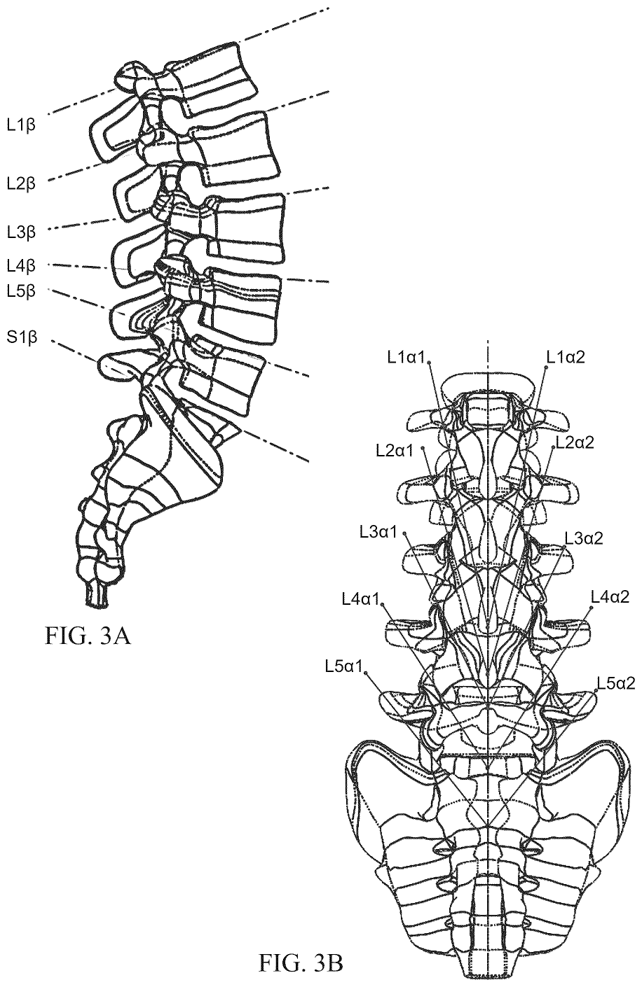

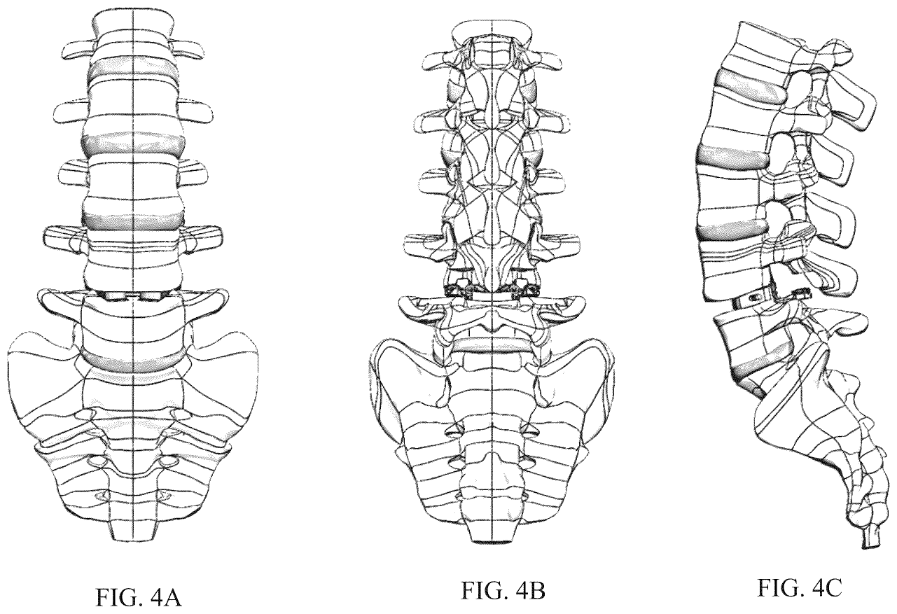

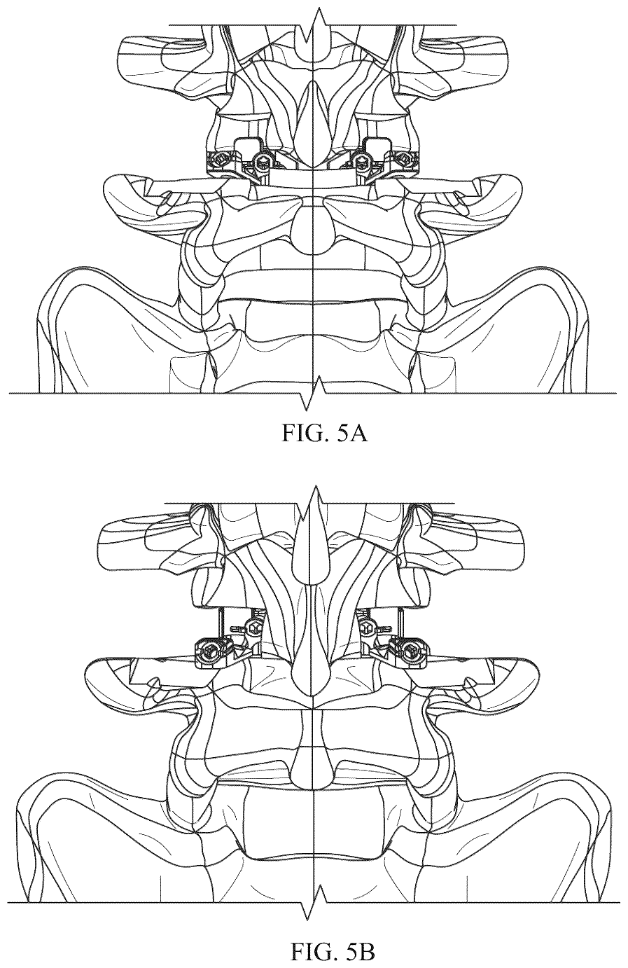

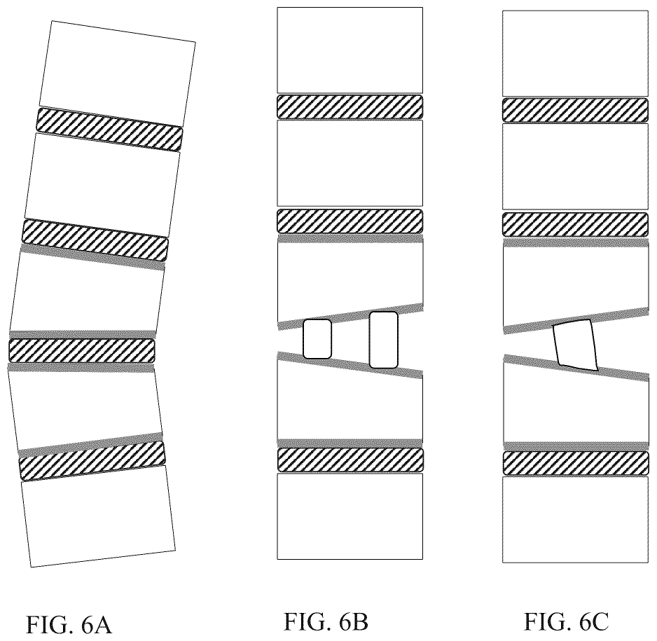

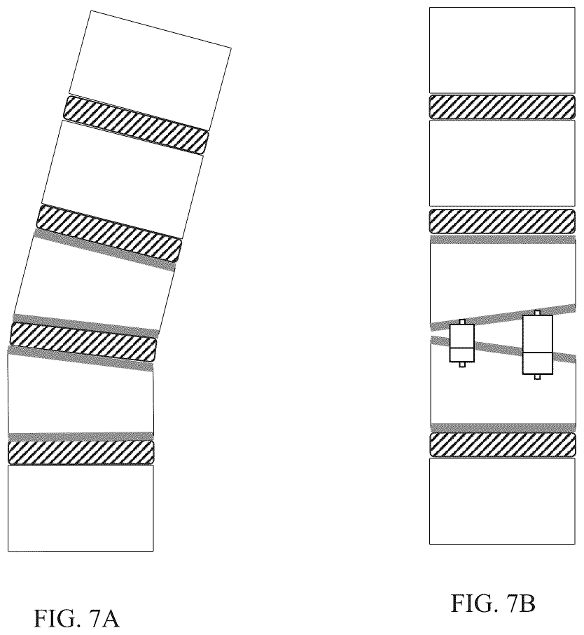

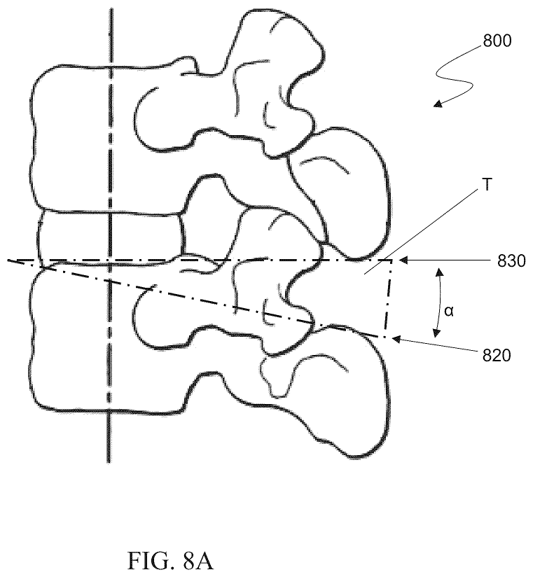

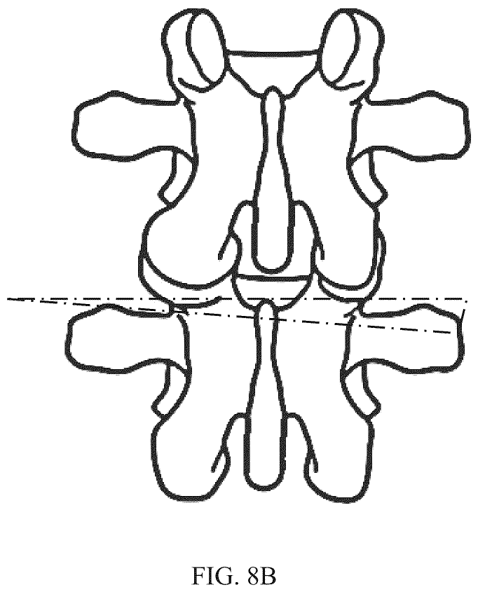

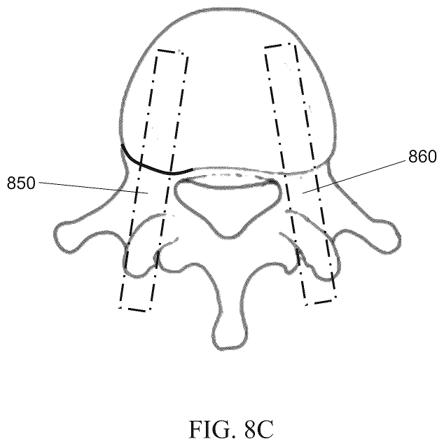

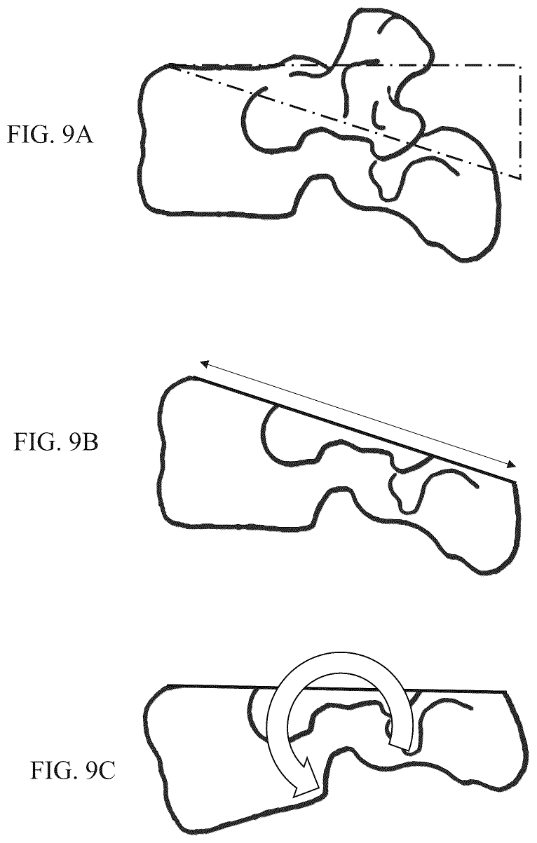

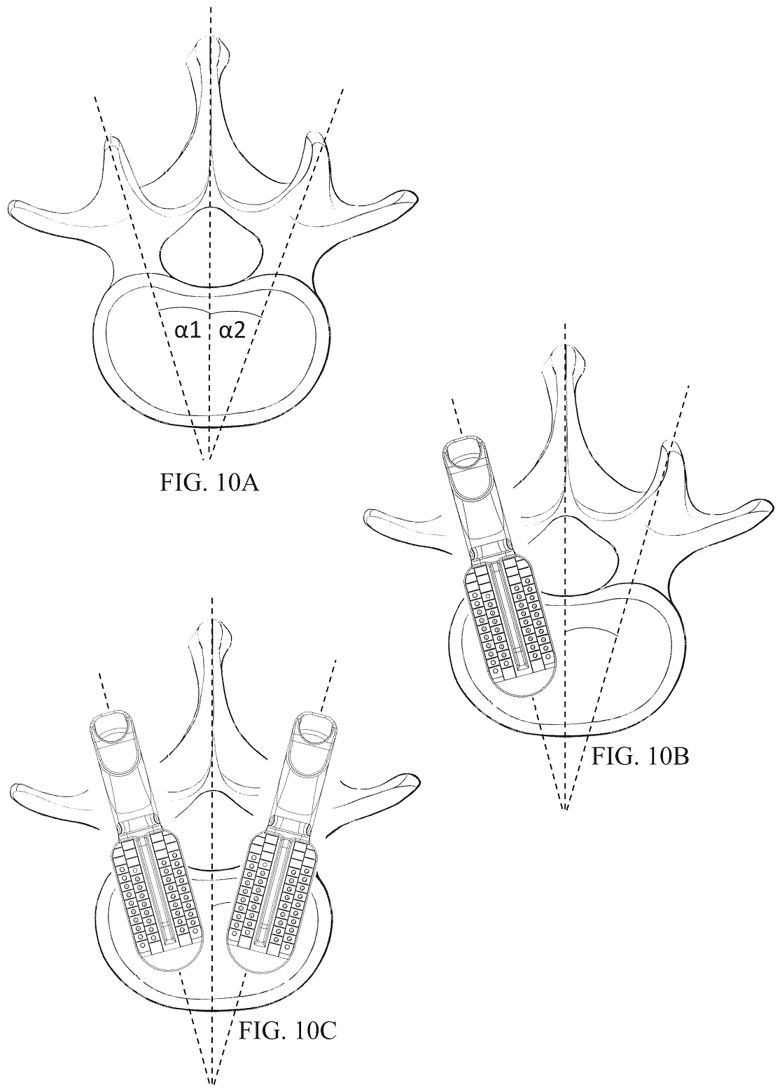

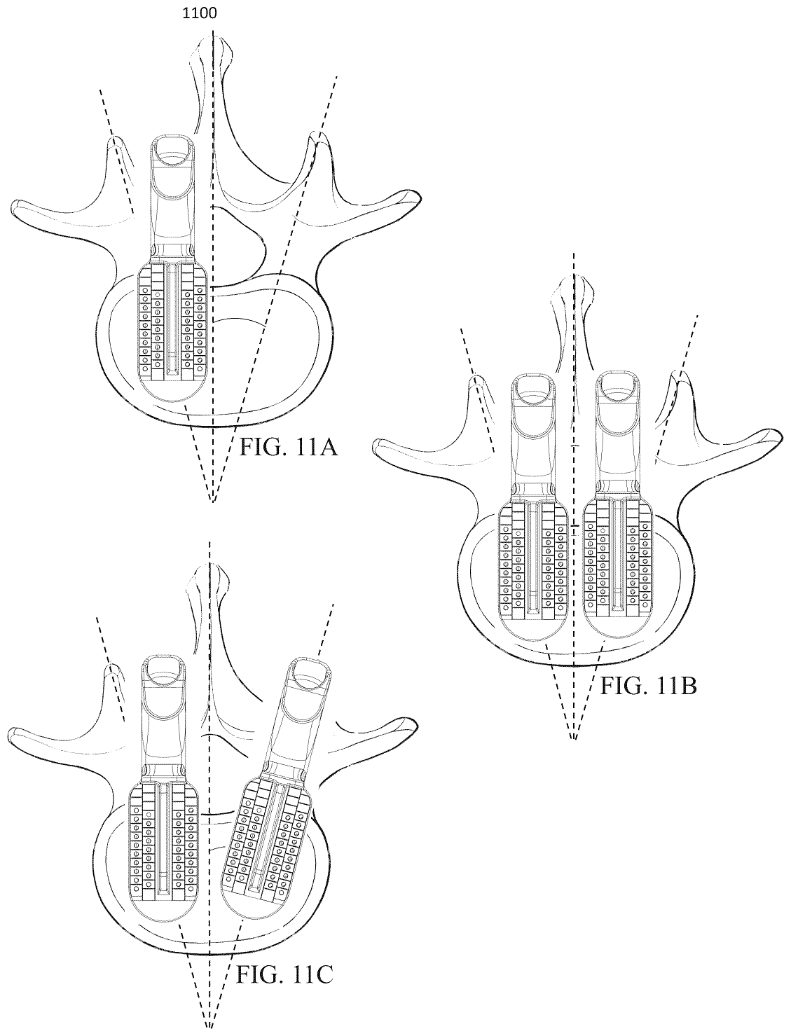

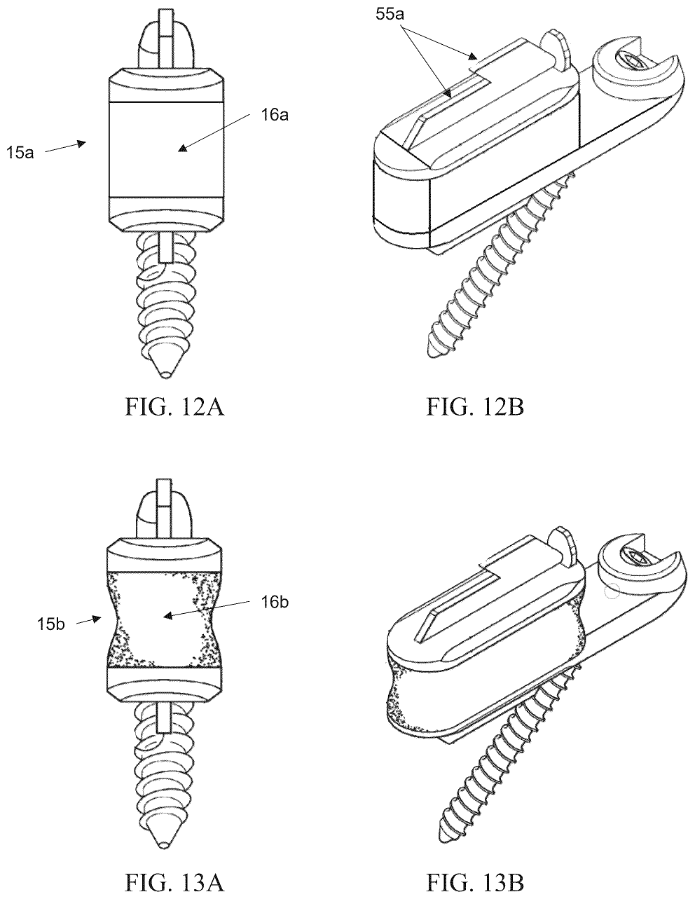

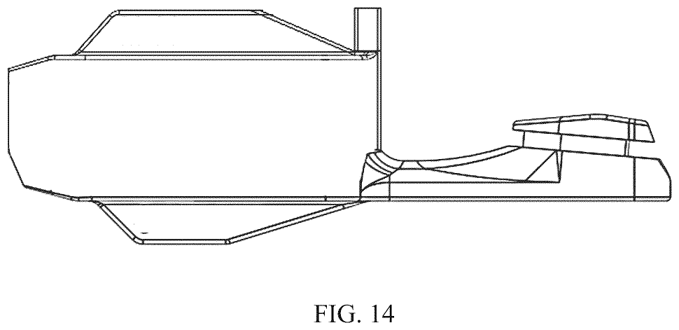

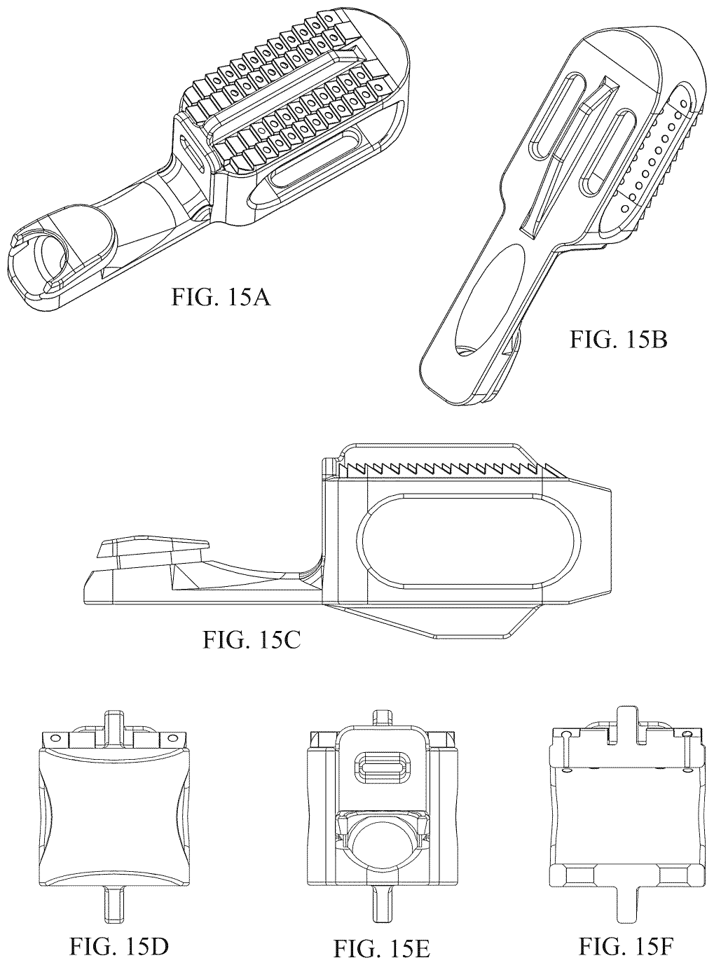

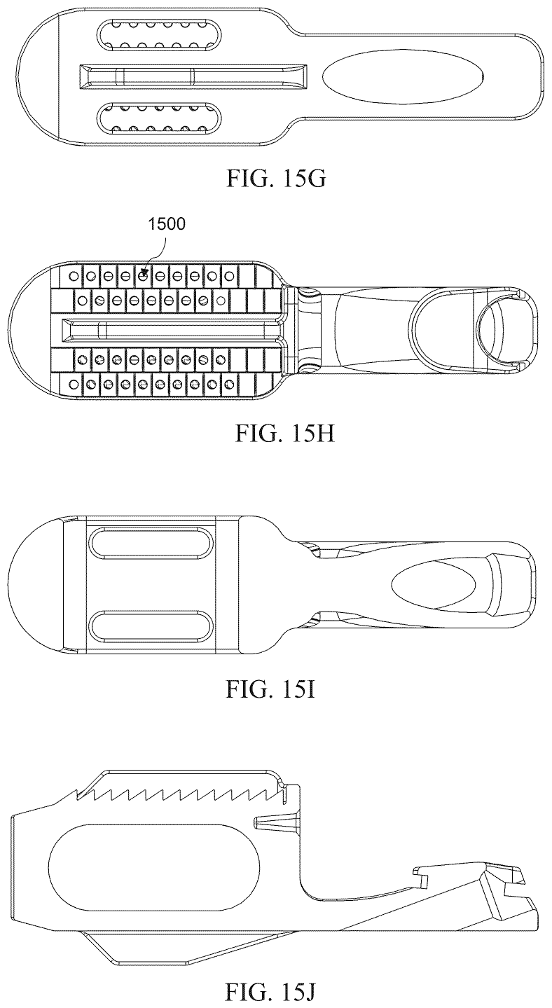

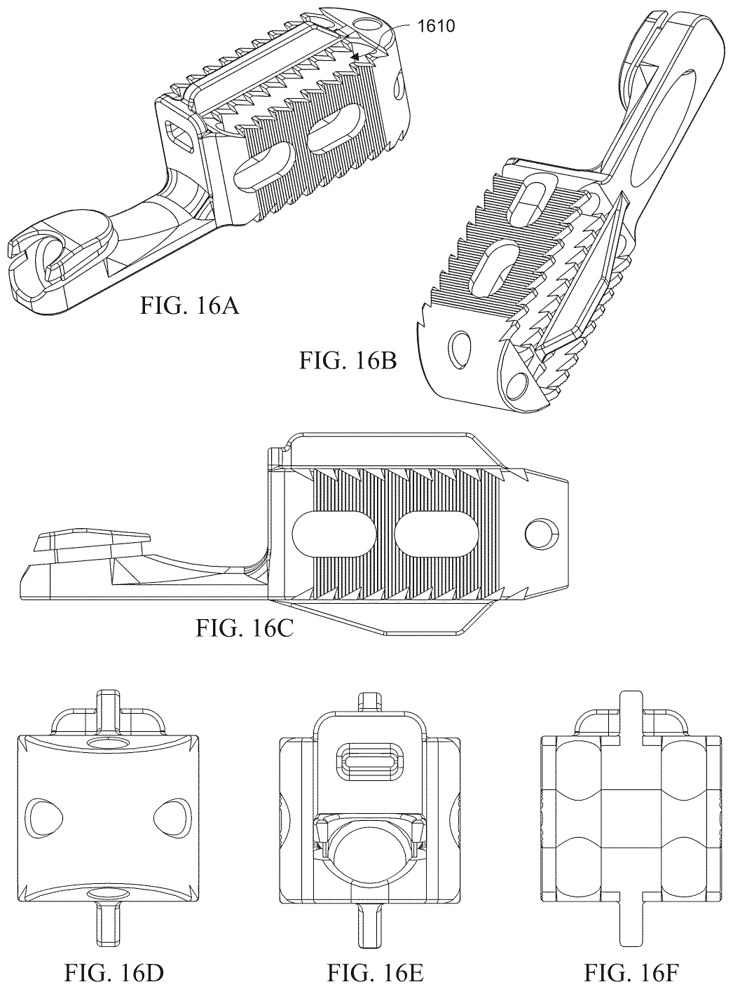

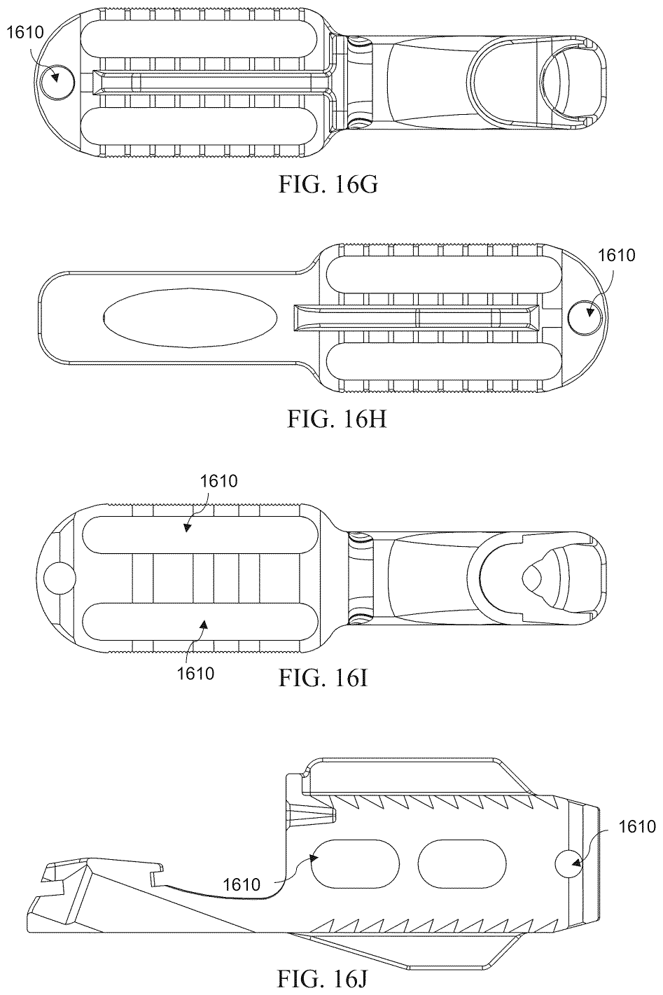

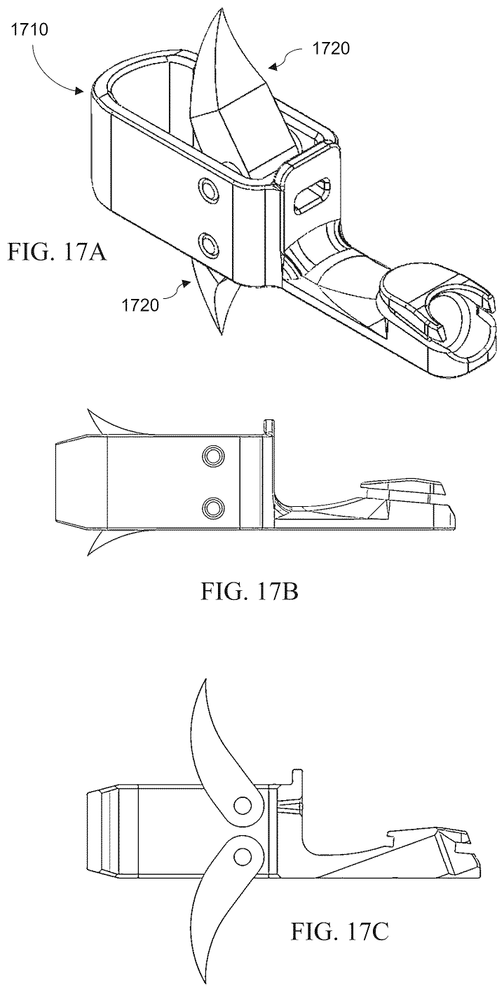

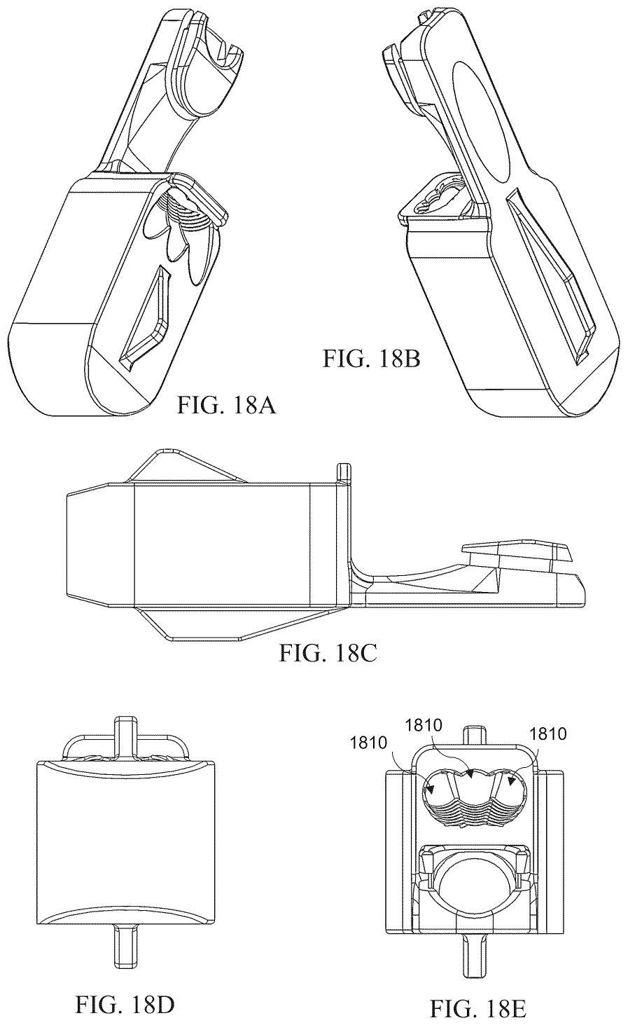

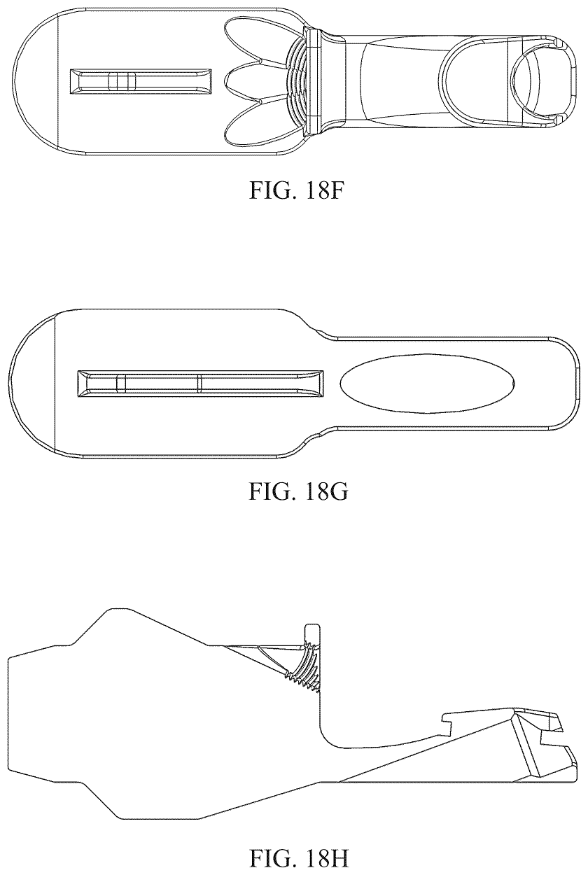

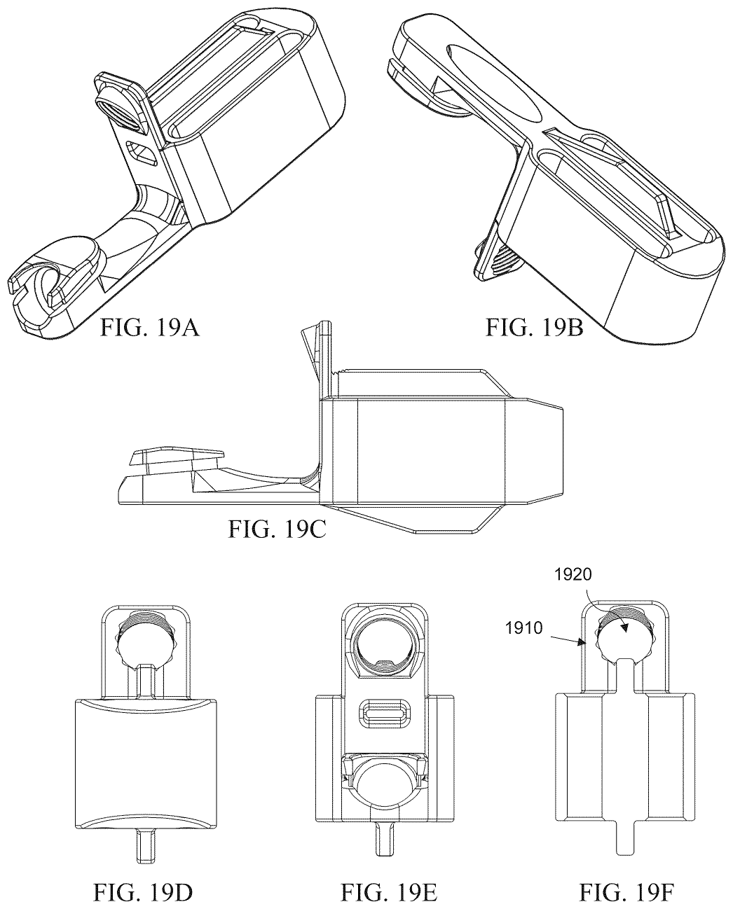

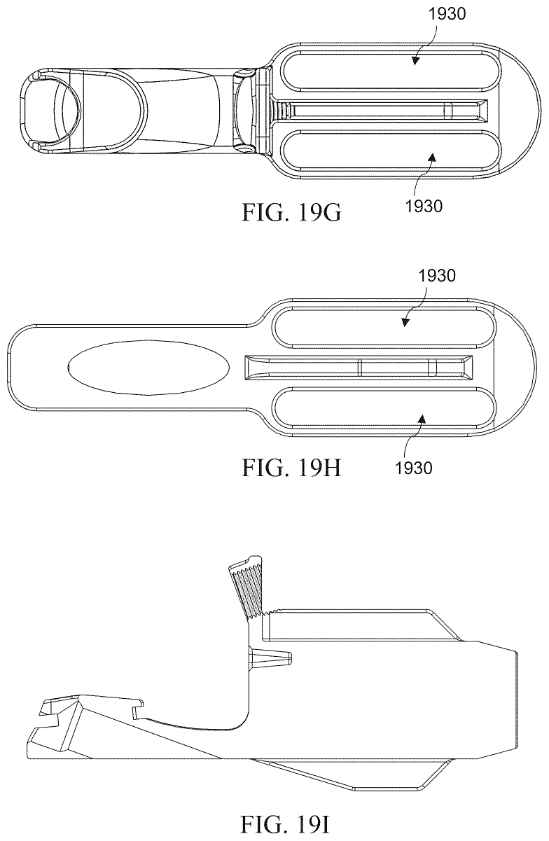

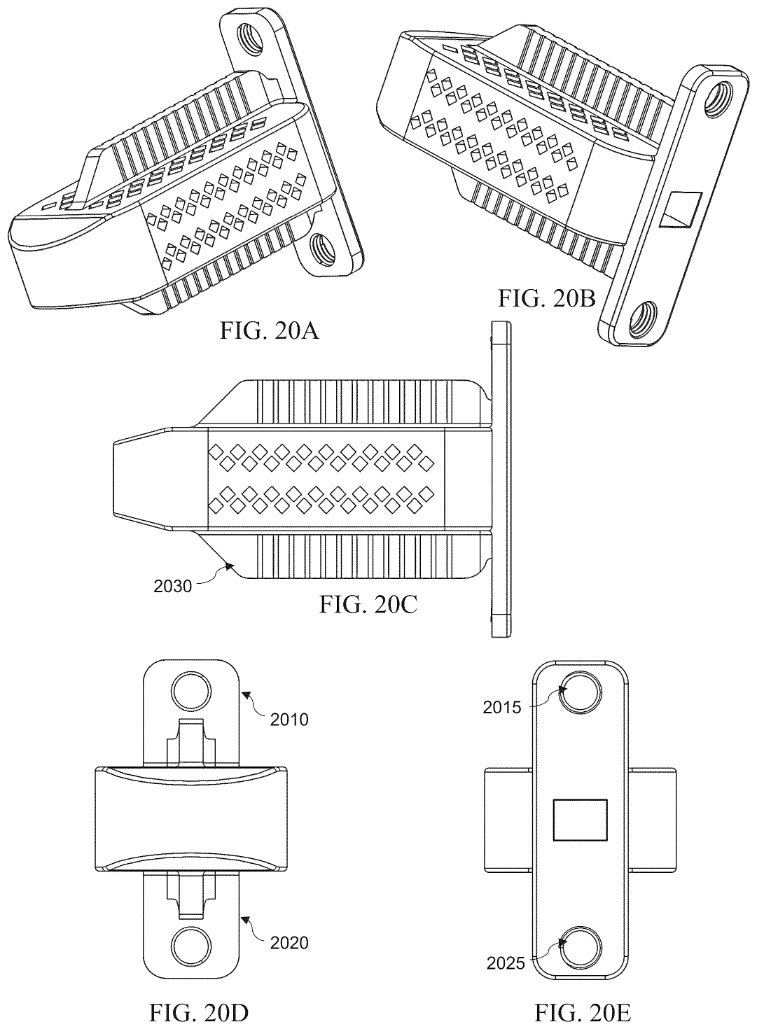

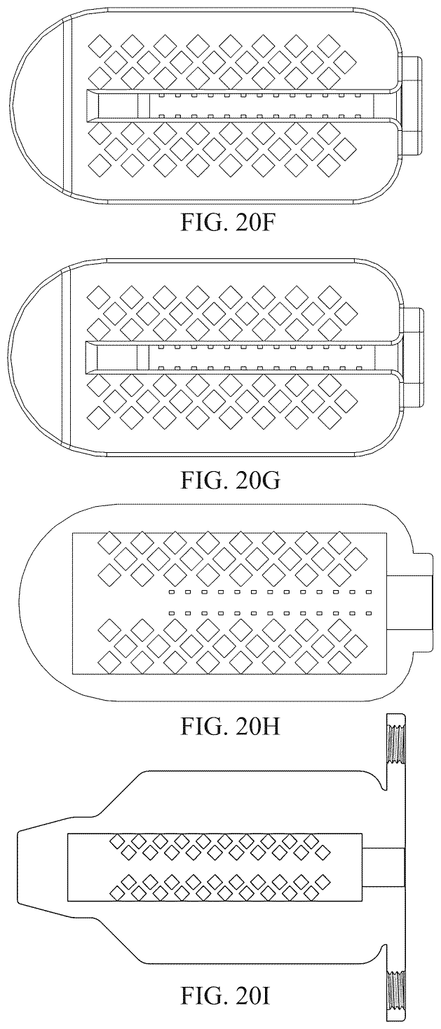

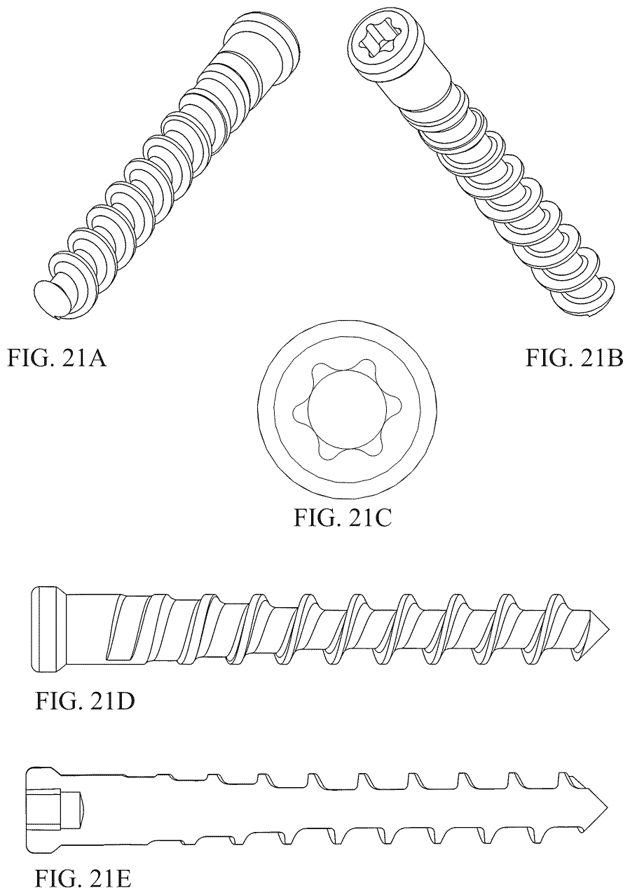

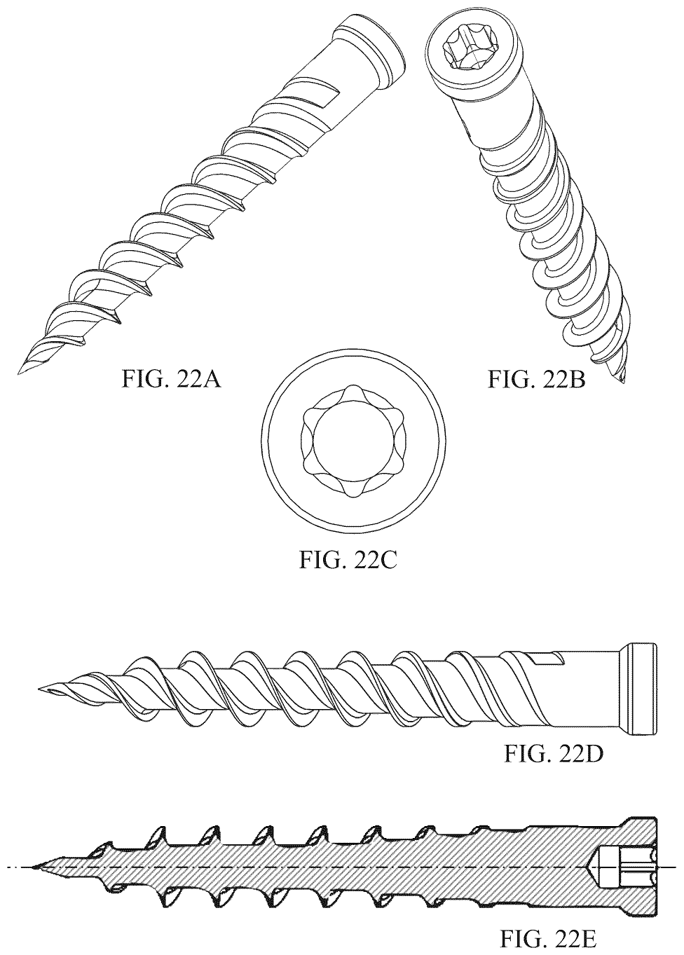

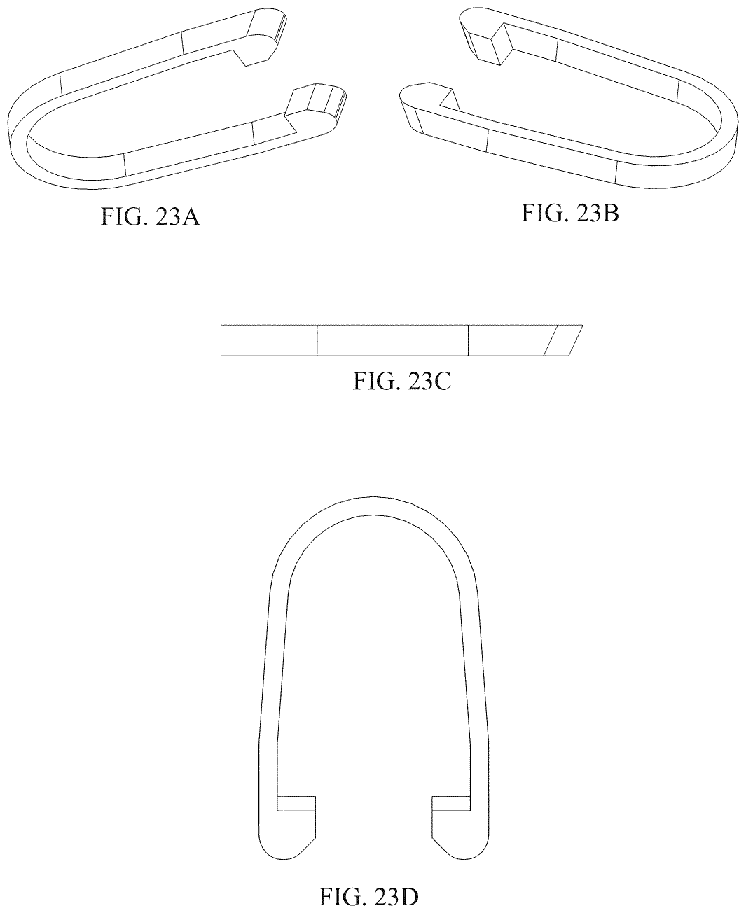

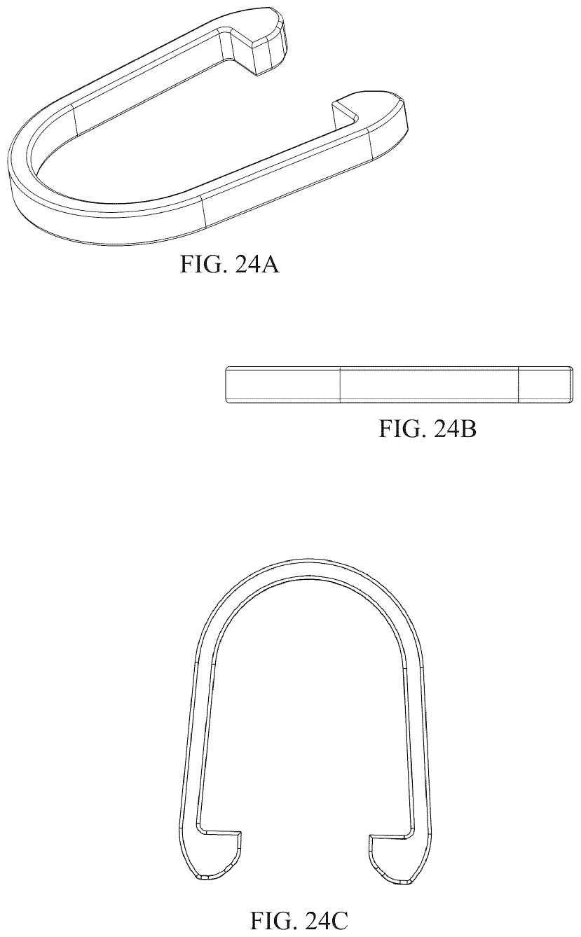

FIGS. 21A through 21E depicts various views of one exemplary embodiment of a fixation screw;

FIGS. 22A through 22E depicts various views of an alternative embodiment of a fixation screw;

FIGS. 23A through 23D depict various views of one exemplary embodiment of a retainer clip; and

FIGS. 24A through 24C depict various views of an alternative embodiment of a retainer clip.

### DETAILED DESCRIPTION OF THE INVENTION

Lumbar fusion is a common surgical procedure for the management of degenerative and spinal deformities. Loss of lordosis after lumbar spine fusion can lead to chronic low back pain, sagittal unbalance, adjacent segment degeneration and other compensatory mechanisms (e.g., knee flexion, hip extension, etc.) Identification and restoration of adequate lumbar lordosis for sagittal balance (and/or other spinal alignment corrections) should be one optimal strategy for the spine surgeon. Optimal lordosis and/or other spinal alignment is different for each individual and depends on the precise measurement of spinopelvic parameters for each individual. Once the proper spinopelvic parameters are obtained, one or more spinal implants can be deployed into one or more vertebral segments of one or more spine regions. Also, bone filler materials may be added to the implants and/or the intervertebral spaces to stimulate bone healing. This desirably results in a permanent or temporary fixation (joining) of a single dysfunctional spinal motion vertebral segment together and/or multiple dysfunctional spinal motion vertebral segments in a spine region, while further restoring proper disc height, restoring of optimal lordosis and sagittal balance.

One significant limitation of traditional spinal arthrodesis procedures (e.g., “spinal fusion”) is the existing need for a treating surgeon to maintain the integrity of the vertebral endplates adjacent to a treated spinal disk space during a fusion procedure. Traditionally, a spinal fusion procedure involves the surgical access and removal of some or all of an intervertebral spinal disk (e.g., the nucleus pulposus and/or some inner portion of the annuus fibrosus) to create an evacuated disk space, and scraping of the adjacent endplates (e.g., above and below the disk space) to promote punctuate bleeding or “bleeding bone” regions above and below the removed disk material. In most interbody fusion cases, one or more interbody implants or “cages” are inserted between the endplates at the bleeding bone regions, and graft material is inserted into the cage(s) and into the remainder of the evacuated disk space. The one or more cages desirably serve as “space holders” between the treated vertebrae, maintaining separation between and relative positioning of the upper and lower vertebrae (often in conjunction with a tension force induced by the remaining annular disk tissues) and promoting bone growth therebetween (e.g., to desirably fuse the vertebral bodies together into an inflexible bony mass).

The insertion of interbody cages into the intervertebral disc space aims at providing mechanical stability through an implant made from a strong material, and at promoting fusion through the autogenous bone graft included in the cage. In order to properly maintain separation between the treated vertebral bodies, and promote bony fusion therebetween, the interbody cage(s) implanted into the evacuated disk space will desirably maintain its/their position relative to each of the vertebral bodies against which it/they sit. More importantly, the one or more cages will desirably not sink or “subside” into one or both of the adjacent vertebral bodies, as such implant subsidence would allow the vertebral bodies to move relative to each other, typically closer together as the implant “sinks” into the endplate surface, which inhibits the desired fusion and often causes severe incapacitating pain to the patient as well as altering the desired spinal alignment and spacing, which can compromise any indirect decompression of nervous structures. However, because blood flow from the endplates can greatly enhance spinal fusion, some portions of the endplates are typically scraped or otherwise “damaged” to induce “bleeding bone” of the endplates—which can often weaken the endplate, especially if too much of the endplate surface is scraped and/or removed. Currently, different fusion cage designs require different endplate preparations. Basically, two techniques can be distinguished: one includes deliberate endplate cavitation to provide a host bed of bleeding cancellous bone; the second technique involves excision of the cartilage endplate down to the preserved, bleeding subchondral bone. To enable fusion, a sufficient amount of potentially osteogenic cells is typically necessary; therefore, bleeding bone is desirably present next to the graft—but in either well accepted technique the endplates of the vertebral bodies are desirably intact.

The shape, density and the strength of a vertebral endplate structure has been shown to vary across its surface, with the center of the endplate being the thinnest and weakest area. It is known that the bony end plate thickness of vertebral bodies varies depending on spinal level and location and is generally between 0.2 and 0.8 mm thick. At a given lumbar level, the bony end plates are thinner centrally than peripherally; also, the end plate cranial to a particular disk is thicker and has higher bone mineral density than does the end plate caudal to it.

In various embodiments, a spinal fusion device may be implanted into a functional spinal unit for a variety of reasons, including to restore stability to a significantly degraded and/or unstable spinal level. If desired, fusion implants could be implanted bilaterally in a treated spinal level, with the implant(s) including a support body to occupy a portion of the disk space (optionally having one or more voids for graft placement to promote bony integration with the adjacent vertebral surfaces and/or an open graft window and/or side ports for graft cell placement, ingrowth and/or on-growth) and a bridge or “tail” for securing to one or more prepared pedicle surface(s) (e.g., to prevent subsidence and/or to cross the foramen), and one or more screw or anchor retention feature(s) for accommodating anchoring screw fixation to one or both adjacent vertebral bodies. If desired, such a device could optionally include radiopaque markers or other features to allow the position and/or orientation of the implant to be monitored in a non-invasive manner after surgery.

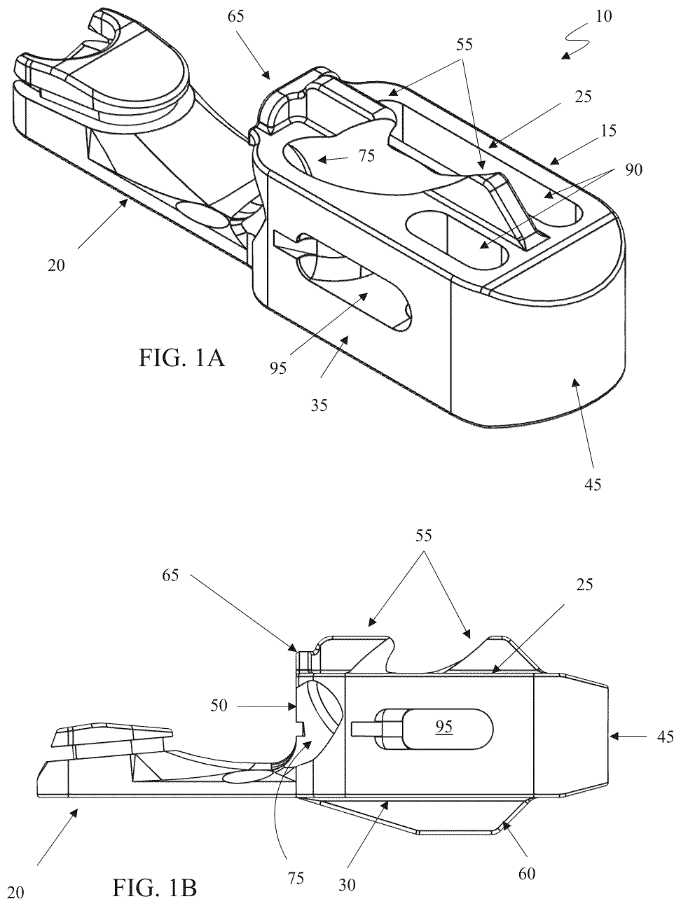

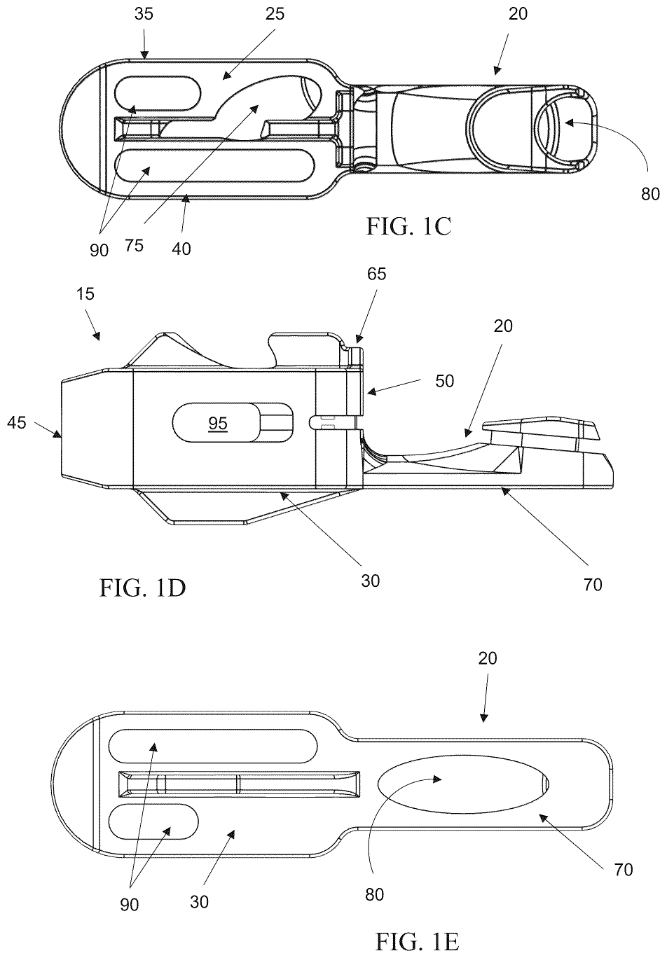

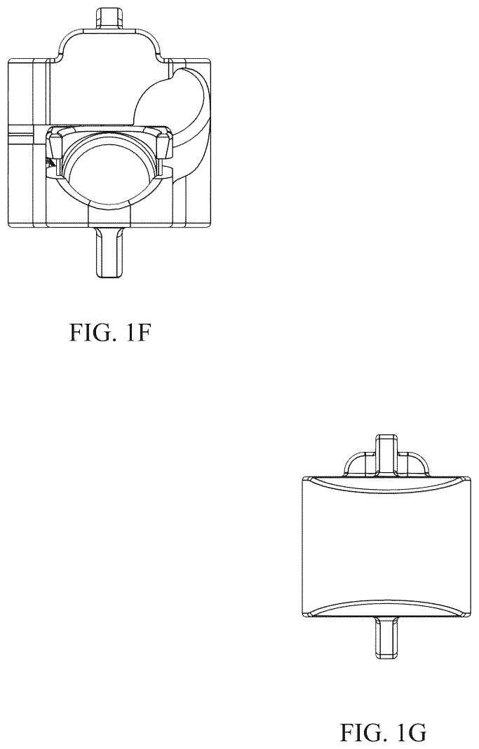

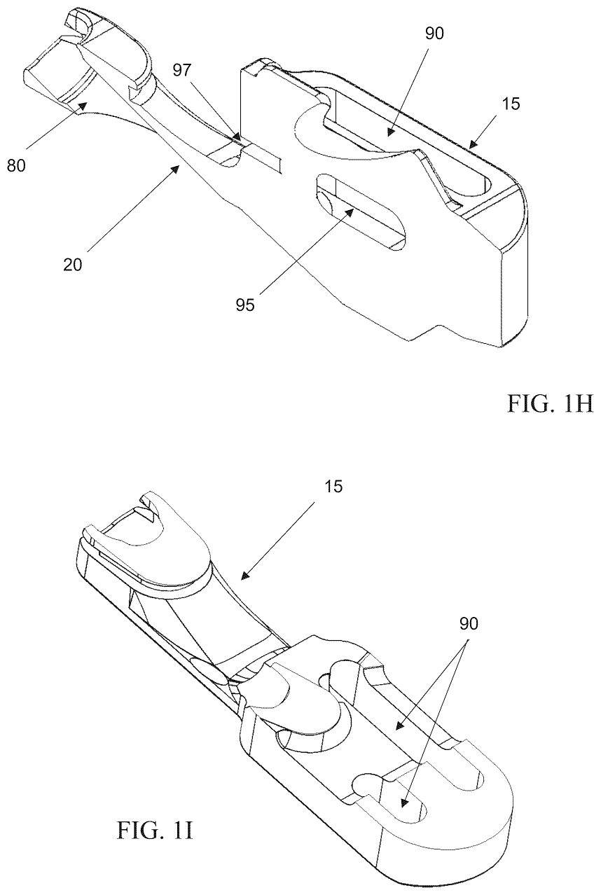

FIGS. 1A through 1G depict various views of one exemplary embodiment of an interbody implant 10 constructed in accordance with various teachings of the present disclosure. The implant 10, which is depicted as a left-sided implant, can include a body 15 and a tail section 20. The body 15 can include a top surface 25, a bottom surface 30, a first side surface 35 (e.g., medial side surface) and a second side surface 40 (e.g., a lateral side surface), an anterior surface 45 and a posterior surface 50. Desirably, the top and bottom surfaces 25 and 30 are intended to contact the endplates or other portions of the adjacent vertebral bodies (not shown), and these surfaces can optionally incorporate textured or coated surfaces that desirably promote bony ingrowth. The top surface 25 can include an upper keel 55 (depicted in two sections in this embodiment) and the bottom surface 30 can include a lower keel 60. The anterior surface 45 can include a curved or tapered profile, and the posterior surface 50 can include an upper tab 65. The tail section 20 will desirably extend from a lower portion of the posterior surface 50, with a bottom tail surface 70 that can optionally contact posterior bone surface of the lower vertebral body (not shown).

An upper fixation opening 75 (e.g., to accommodate a bony fixation device such as a bone screw-not shown) is formed in the body 15 which extends through a portion of the top surface 25, and a lower fixation opening 80 is formed in the tail section 20 which extends through a portion of the bottom tail surface 70. In addition, one or more openings or pockets 90 can be formed into and/or through the body 15, which can desirably be filled with bone graft or other materials (not shown) which desirably promote bone growth into and/or through the pockets 90 to secure the adjacent vertebral bodies with bony fixation (e.g., arthrodesis) opening top surface 25 In the exemplary embodiment, a transverse opening 95 extends from the first side surface to the second side surface (e.g., a medial to lateral opening), which may be utilized to accommodate bone graft and/or to view the implant to determine if an effective arthrodesis has been formed post-surgery.

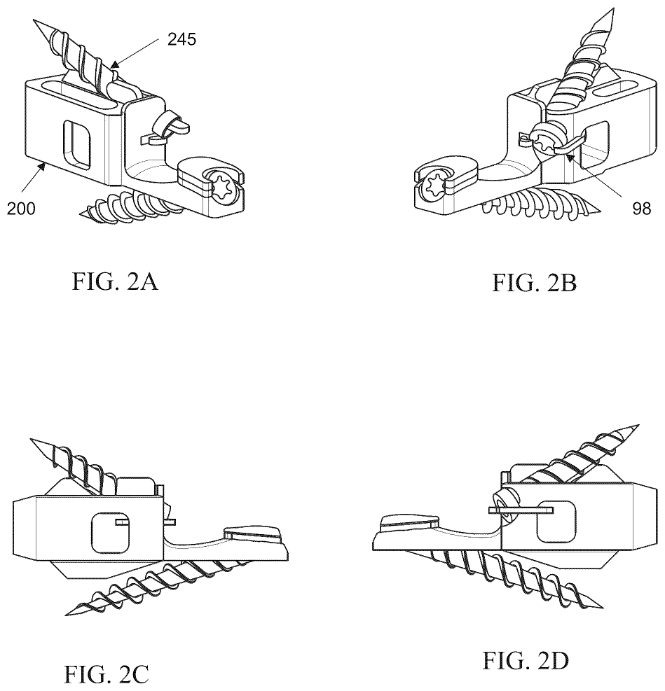

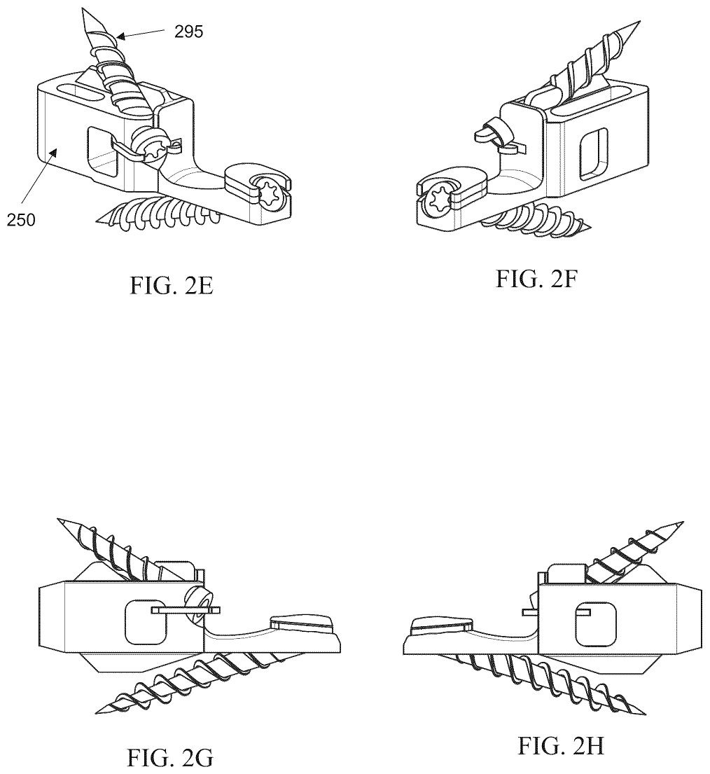

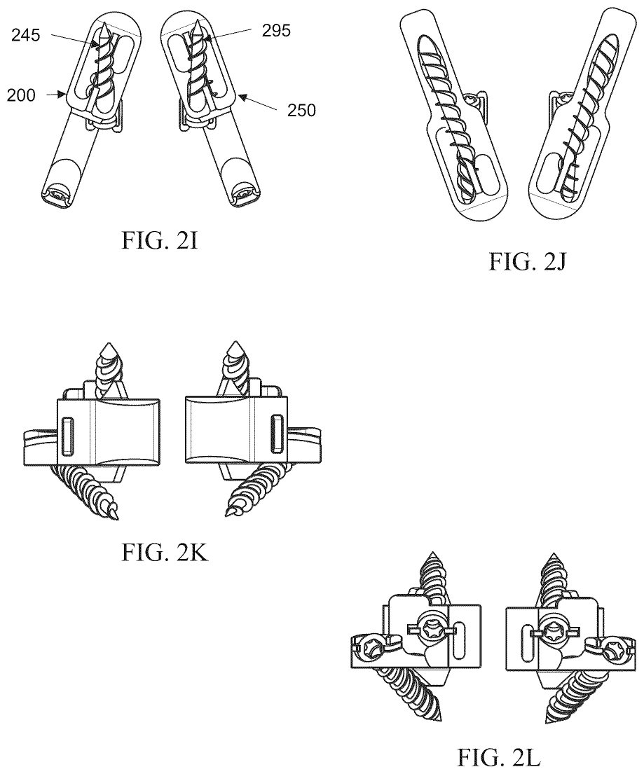

FIGS. 1H and 1I depict cross-sectional views of the interbody implant of FIGS. 1A through 1G. FIG. 1H is a vertical A/P planar view of the implant, and FIG. 1I is a horizontal planar view of the implant. As best seen in FIG. 1H, the lower fixation opening 80 extends through a portion of the tail section 20, and the upper fixation opening 75 extends through the body 15, with each of these openings desirably sized and configured to accommodate a fixation screw or other fixation element which secures the implant to the adjacent vertebrae. As best seen in FIG. 1I, the transverse opening 95 (e.g., which extends through the body 15 from side to side) can optionally pass through the one or more pockets 90, with the disclosed pockets extending generally vertically and/or from the top to the bottom surface of the body 15. Also depicted is a groove 97 for accommodating a securement clip 98 (see FIG. 2B), which clip can engage a head of a fixation screw to desirably prevent screw loosening or dislodgement.

FIGS. 2A through 2L depict exemplary embodiments of complimentary interbody implants, wherein FIGS. 2A through 2D depict various view of the left-side interbody device of FIGS. 1A through 1G with fixation screws attached, FIGS. 2E through 2H depict various view of a right-side interbody device 250 with fixation screws attached, and FIGS. 2I through 2L depict various view of the left-and right-side devices and associated fixation screws in one exemplary positioning, such as could occur inside of an intervertebral space.

As best seen in FIGS. 2A and 2B, the upper fixation screw 245 of the left-side interbody device 200 includes an upper fixation screw opening which cants to a left side of the implant, while the upper fixation screw opening of the right-side interbody device 250 cants to a right side of the implant. As best seen in FIG. 2L, this arrangement desirably facilitates placement of the left-side interbody device 200 and the right-side interbody device 250 along respective pedicle axes, while the upper fixation screws 245 and 295 of the two implants can be substantially aligned along an A/P plane. This arrangement desirably provides maximum fixation for the implants, as the upper fixation screws 245 and 295 will desirably pass adjacent to and/or through cortical rim structures of the upper vertebral bodies, providing an extremely strong anchoring and support point for the implant, even where significant removal of cortical endplate material from the upper vertebral body has occurred (e.g., these structures might provide sufficient support for the fusion implant even when partial and/or total endplate removal from the upper vertebral body has occurred). Of course, due to anatomical variability and/or surgeon preference, the implants may not be positioned exactly along the axes shown, or even in symmetry.

In any of the described embodiments herein, bone contacting surfaces of the prosthetic device can including contact surfaces, keels, and/or any bridge surfaces may include features or coatings which enhance the fixation of the implanted prosthesis. For example, the surfaces may be roughened such as by chemical etching, bead-blasting, sanding, grinding, serrating, and/or diamond-cutting. All or a portion of the bone contacting surfaces of the prosthetic device may also be coated with a biocompatible and osteoconductive material such as hydroxyapatite (HA), tricalcium phosphate (TCP), and/or calcium carbonate to promote bone in growth and fixation. Alternatively, osteoinductive coatings, such as proteins from transforming growth factor (TGF) beta superfamily, or bone-morphogenic proteins, such as BMP2 or BMP7, may be used. Other suitable features may include spikes, ridges, and/or other surface textures. Spinal Implant Placement

One primary goal of fusion surgery is to relieve pain, numbness, tingling sensations, weakness, restore nerve function, and stop or prevent abnormal motion in the targeted spinal segment in a spine region. This is desirably accomplished by fusing (e.g., joining) a single dysfunctional spinal motion vertebral segment together and/or multiple dysfunctional spinal motion vertebral segments in a spine region with a spinal implant and/or other mechanical or biological fusion devices or fixtures. Fusing the one or more dysfunctional spinal motion vertebral segments together should ultimately stabilize and align the spine, restore the normal disc space between the bones, and prevent further damage to the spinal nerves and cord.

Spinal fusion surgery options can be performed via a wide variety of surgical approaches, including by posterior lumbar interbody fusion (PLIF), transforaminal lumbar interbody fusion (TLIF), minimally invasive transforaminal lumbar interbody fusion (MI-TLIF), oblique lumbar interbody fusion/anterior to psoas (OLIF/ATP), lateral lumbar interbody fusion (LLIF) and anterior lumbar interbody fusion (ALIF), and/or combined circumferential (360 degrees) approaches. Intervertebral disc material may be removed and are prepared to expose bone and/or a resected bone surfaces. The shape and size of the spinal implant vary with the intervertebral space available to surgically insert them. Once the spinal implants are deployed, the “fusing” or connecting of one or more dysfunctional spinal motion vertebral segments may occur through osseous spinal fusion that is further attained by the use of bone grafts or other osseous methods in the vascularized tissue bed.

FIGS. 3A and 3B depict a sagittal view of an exemplary lower lumbar spinal segment and a posterior view of a lower lumbar spinal segment. In order to realign the spine and/or restore a desired or normal disc space between the one or more vertebral segments, surgeons may evaluate each patient's current status and average patient lordosis, sagittal alignment and/or coronal alignment. Sagittal and/or coronal alignment of the spine is an important consideration as achieving proper lordosis and/or other alignment(s) can dramatically improve the outcome of a given spinal fusion procedure, and in many cases can reduce and/or prevent the occurrence of adjacent segment diseases and improve long-term success of fusion.

FIG. 3A illustrates a typical lumbar lordotic angular variance across various spinal levels indicated by dotted lines. The spine's natural lordotic and kyphotic curvatures and its angular variance are designed for even distribution of weight and flexibility of movement. These natural curves work in harmony to keep the body's center of gravity aligned over the hips and pelvis. For example, as discussed in “Lumbar Lordosis: A Study Of Angle Values And Of Vertebral Bodies And Intervertebral Discs Role” by Fonseca Damasceno et al, published in Acta Orthopedica Brasileira, (ISSN 1809-4406), as compared to the sacral angle, L1β normally has a typical lumbar lordosis angular range of 14 degrees to −9 degrees (14°:−9°), L2β has a typical angular range of 7 degrees to −8 degrees (7°:−8°), L3β has a typical angular range of 14 degrees to −9 degrees (14°/−9°), L4β has a typical angular range of 4 degrees to −14 degrees (4°:−14°) and L5β has a typical lumbar angular range of 0 degrees to −19 degrees (54:0°:−19°). It is desirable to restore the spine to adequate or optimal lordosis or kyphosis as a primary surgical strategy to prevent adjacent segment disease and/or changes of load on different structures within the spine.

FIG. 3B depicts a posterior view of the lumbar spinal segments showing typical right and left transverse pedicle angles (TPA) for one or more spinal segments. The transverse pedicle angles (TPA), the pedicle width, pedicle length, the pedicle height or diameter, and/or the transverse sagittal pedicle angle and may vary between each vertebral spinal segment or each vertebral functional spinal unit as disclosed in “Thoracic and Lumbar Vertebrae Morphology in Lenke Type 1 Female Adolescent Idiopathic Scoliosis Patients” by Xiobang Hu, MD, PHD et al, Int. J. Spine Surg. (2014) 8:30; “Morphometry of the Lower Thoracic and Lumbar Pedicles and its Relevance in Pedicle Fixation, “S.P. Mohanty et al., Musculoskeletal Surgery (2018) 102:299-305; and “A Comparison of Lumbar Transverse Pedicle Angles between Ethnic Groups: A Retrospective Review,” by Robert Stockton et al., BMC Musculoskeletal Disorders (2019) 20:114, which are herein incorporated by reference in their entireties. Moreover, the understanding of the morphology and/or the anatomical parameters of the vertebral body within one or more segments of the spine and the anatomic relationship of the pedicles and adjacent neural structures may help reduce pedicle screw and spinal implant malposition, as well as increase strength, stiffness and support of the vertebral body to decrease postoperative complications and pain sensation.

FIGS. 4A through 4C, 5A and 5B depict an anterior view, a sagittal view and a posterior view, respectively, of an exemplary spine segment within a spine region wherein a pair of spinal fusion implants such as disclosed herein have been implanted into a single intervertebral segment. In various embodiments, the treatment can include accessing a targeted spinal level and removing some disc material to create an intervertebral space between an upper or superior vertebra and a lower or inferior vertebra. The treated region may include cervical, thoracic, lumbar and/or sacral regions of the spine. In one embodiment, a first implant can be implanted via a first Kambin's triangle access (e.g., adjacent a first pedicle) at a first orientation relative to a midline of the lower vertebral body on a first side of the intervertebral space, and a second implant is implanted via a second Kambin's triangle access (e.g., adjacent the second pedicle) at a second orientation relative to a midline of the lower vertebral body on a second side of the intervertebral space. The intervertebral space may be a single space created by removing the intervertebral disk and/or other structures, or separate intervertebral spaces (e.g., separate medial and lateral spaces) may be created to accommodate the first and second implants.

In various embodiments, the first and second orientations may be mirror images of each other, and/or may substantially align and/or follow along the transverse pedicle angle of at least one side, e.g., a right side and/or a left side, which may include asymmetric placement. In other embodiments, only a single implant may align or follow along a transverse pedicle angle on a first side, such as a right side and/or a left side, with the other implant at virtually other orientation. Of course, in some embodiments only a single implant on a single side of the vertebral body may be implanted, if desired.

The one or more implants placed at a single vertebral level may comprise the same implant height or differing implant heights, as well as comprising the same lengths and/or different lengths. In various embodiment a first orientation and/or a second orientation may comprise the same or differing angles, including angles ranging from 0 degrees to 60 degrees and/or 60 degrees or less.

One particularly beneficial aspect of the present invention and related techniques is the ability of the tail section of the implant construct, and more particularly the bottom tail surface in combination with the fixation afforded by the lower fixation opening, to stabilize and support the majority and/or entirety of the implant in a variety of support conditions, including where little or no of the superior endplate of the lower vertebral body is retained beneath the implant during the surgical procedure. Traditionally, where retention of the natural vertebral body was desired or mandated (which is the typical surgical case), a treating surgeon was forced to maintain the integrity of the vertebral endplates adjacent to a treated spinal disk space during a fusion procedure, as removal of too much endplate material would likely allow the intervertebral implant to migrate or “sink into” the softer cancellous bone of the vertebral interior. Accordingly, where any adjustment of the intervertebral alignment between adjacent vertebral bodies was desired for a damaged or diseased spine (see FIG. 6A), the surgeon would be forced to use standard implants which altered the spacing and/or angulation between the existing endplate anatomy, such as the use of two interbody implants of different sizes (see FIG. 6B) or a single implant or graft potentially particularized for the patient's anatomical condition (see FIG. 6C).

In contrast to existing techniques and surgical requirements, the disclosed implants desirably allow a surgeon to partially and/or completely remove some or all of the lower endplate material and/or depress various of the lower implant surfaces into the cancellous bone of the lower vertebrae, wherein lower vertebral support for the implant can be primarily provided by the posterior elements of the lower vertebral body (e.g., the pedicular structures and/or other posterior support structures of the vertebral body), which may further supported by posterior rim structures and/or any remaining anatomy of the lower vertebral body (e.g., via the fixation screw). Moreover, the ability to remove and/or modify such structures grants a surgeon the ability to significantly alter the alignment between the upper and lower vertebral bodies, including changes to the relative vertebral spacing, the anterior/posterior positioning and alignment, the medial/lateral positioning and alignment, and/or various combinations thereof.

In addition to improved lower vertebral support, various of the embodiments disclosed herein provide for contact between an upper surface of the fusion implant and the posterior cortical rim of the superior vertebral body, and in some embodiments the fusion implant is directly secured to the superior vertebral body using one or more fixation screws or similar devices which can be secured through a portion of the cortical rim of the vertebral body. This mode of fixation can provide for a significantly stronger fixation between the upper vertebral body and the disclosed system components than available for traditional fusion implants, as such prior art implants have not been typically implanted at or near the periphery/rim of the endplate for a variety of reasons, including the significant opportunity for implant migration to result in unwanted contact between the prior art implant and the spinal cord or other neurological/anatomical structures. In contrast, the disclosed devices can be supported by the periphery/rim of the endplate, which are typically extremely strong structures for such fixation.

FIG. 8A depicts a side view of one exemplary spinal motion unit 800 that is undergoing a surgical procedure in accordance with one exemplary embodiment of the present invention. In this embodiment, preoperative image data of the spinal motion unit has been obtained, and a surgical plan to alter the alignment of the spinal motion has being proposed. In this embodiment, a proposed lower component alignment path 820 has been presented, which will desirably result in the surgical removal of a “wedge” of bony material from the lower vertebral body and/or one or both pedicles, which is represented by the shaded triangle “T” of FIG. 8A (involving removal of bony material at or below the anatomical alignment line 830 up to the revised alignment line of 820). Desirably, this surgical plan will allow some and/or all of at least the bottom of the pedicles to be preserved during such removal, such that the remaining portions of the pedicle remain attached to the vertebral body, which can desirably provide additional stability to lower surfaces of the implant. If desired, the resection may be symmetrical on each side of the vertebral body, or the resection may be asymmetrical in some fashion.

FIG. 8B depicts a posterior view of the exemplary spinal motion unit, where an asymmetrical resection is being planned to desirably correct an undesirable medial/lateral curvature of the spine. In this embodiment, more material can be resected from right side of the spinal motion unit than from the left side, which will desirably induce a slight medial curvature to the patient's spine (i.e., providing a desired coronal plan correction). In addition, as previously noted, the surgical plan will desirably allow some and/or all of at least the bottom of the pedicles to be preserved during such removal, such that the remaining portions of the pedicle are attached to the vertebral body, to provide additional stability to lower surfaces of the implant.

FIG. 8C depicts a top view of a vertebral body of the surgical plan of FIG. 8A, in which the proposed bone “wedges” are shown in shadow as planning boxes 850 and 860. In this embodiment, the wedges could be taken from both sides for sagittal correction, or both sides asymmetrically or unilaterally for combined coronal and sagittal correction. In addition, the medial/lateral alignment of the left and right implants does not appear symmetrical with relation to the A/P midline of the vertebral body, which the present system can easily accommodate.

FIGS. 9A through 9C depict one exemplary lordotic correction that could be obtained using the teachings of the present invention. In this embodiment, a vertebral body is imaged, and a surgical resection plan is proposed (indicated as the shaded triangle). FIG. 9B shows the vertebral body after resection, and FIG. 9C depicts the new orientation of the vertebral body after resection is complete, which could represent an increased lordotic curvature of the lumbar spine when accomplished at the lumbar level. In one exemplary embodiment, a resulting correction to the alignment between vertebral bodies of a spinal motion segment could comprise altering a negative 4 degree curvature to a positive 14 degree curvature, which could then be fused in position (on one or both sides, including different corrections on each side of the spinal unit) using the various implants disclosed herein.

While the employment of a single implant device at a given spinal level is contemplated herein, it is anticipated that bilateral implantation of a pair of fusion devices at a single spinal level will be a common method of employment for a large number of spinal arthroplasty procedures. For example, a first implant of a plurality of implants can be deployed at a first right orientation into a first right side of a vertebral level and a second implant of the plurality of implants can be deployed at a left orientation into a first left side of a first intervertebral space of a first spine segment of a first spine region. Where multi-level treatments are contemplated, a second plurality of implants can be deployed at a second spinal level in a similar manner. The orientation and or position of each of the spinal implants may be the same or may vary, and may include one or more implants that align or follow along a transverse pedicle angle of at least one side, a right side and/or a left side. The various implants may comprise the same heights and/or lengths or may comprise differing heights and/or lengths.

### Spinal Realignment Concurrent With or Prior to Fusion

Various embodiments disclosed herein include the realization that altering the spinal alignment of one or more spinal levels, such as altering a motion segment to a more normal kinematic alignment and/or modifying spinal alignment in some other desired manner prior to or during a spinal fusion procedure, can greatly improve surgical outcomes. For example, it may be desirous to restore a natural alignment between two diseased and/or degenerated adjacent vertebrae before fusing said vertebrae, which may improve the potential for a successful arthroplasty at the treated level and/or may achieve a more desirable overall alignment of the entire spine and/or motion segments thereof. In some instances, altering a treated spinal level to a condition further away from a natural alignment may be desirous to compensate for malignment of the spine at other motion segments, for example.

Another significant feature of the disclosed invention includes the realization that the surgical alteration of degenerated and/or diseased spinal anatomy to a more normal kinematic positioning or alignment before and/or during implantation of a fusion implant can significantly improve implant performance and/or durability, reducing undesirable loading and/or loading modes from affecting the implant and/or the surrounding anatomy in an undesirable manner, and greatly improve clinical outcomes and/or patient satisfaction. Accordingly, various embodiments encompass spinal implants and related surgical methods and procedures that are designed to stabilize one or more treated spinal levels and/or portions thereof while concurrently achieving a desired spinal alignment. Such stabilization and/or restoration of normal kinematic alignment can dramatically improve surgical outcomes for fusion implant components, as the restoration of balance and/or kinematic motion to the spine or various portions thereof may dramatically reduce loading and/or stresses experienced by the fusion implant components, thereby reducing implant wear, failure and/or implant migration in many cases.

In one exemplary embodiment, a method for restoring alignment to a targeted spinal segment via fusion to one or more locations of the spine can comprise the steps of collecting preoperative data (e.g., two-dimensional and/or three-dimensional data) regarding the anatomy of a patient during a preoperative protocol and/or a pre-operative method, and then analyzing this data in various ways to create a surgical plan for intraoperative repair of the patient's anatomy. The ultimate goals of such a preoperative protocol can be to analyze, estimate, assess and/or predict alignment of the patient's spine and/or spinal segment(s) thereof and to plan a surgical correction which alters various aspects of the spinal alignment to restore a desired alignment to some or all of the patient's spine and/or the targeted spinal segments thereof, with an objective of improving patient outcomes and/or reducing the occurrence of intraoperative/postoperative morbidity or mortality, as well as reduce the overall risk to the patient. More specifically, the detailed goals of the preoperative procedure can include the documentation of the condition(s) for which surgery is required; assessing the patient's overall health status to uncover hidden conditions that could increase perioperative and postoperative risk; collection and analysis of anatomical data, development of an appropriate surgical or intraoperative plan; education of the patient about the upcoming surgery to reduce anxiety; and/or reduce costs by shortening hospital stay and increasing patient satisfaction.

In various embodiments, anatomical data regarding the alignment and motion of the patient spinal anatomy and/or related structures can be collected and analyzed, such as via a preoperative imaging protocol. Preoperative imaging can be an essential tool for providing a current picture and understanding of a patient's condition and morphological bony structures. Acquiring one or more images provides surgeons with more information and the surgeons can exploit the anatomical and functional data to help develop an intraoperative or surgical approach to restore alignment and/or otherwise treat the patient's degenerated condition. One exemplary imaging protocol can comprise the steps of: completing at least one MRI imaging scan to evaluate various soft-tissue related pathology, including disc degeneration grade, facet joint cartilage, and nerve compression; obtaining at least one or more radiographs (e.g., X-rays) at different static and dynamic positions, such positions including at least a standing anterior/posterior, flexion/extension, a lateral neutral, a lateral slump sitting views to evaluate biomechanics and/or any combination thereof. The imaging protocol may further include the step of obtaining at least one CT scan or other imaging modalities.

The imaging protocol may be desirably used to review, analyze and/or understand a patient's bone morphology and spinal biomechanics. The spinal biomechanics may be evaluated during the lordotic changes between standing neutral and lateral sitting, which may be indicative of guarding behaviors, spinal rigidity and/or flexibility and pathology of the patient anatomy. The lateral sitting view may also be used to evaluate degree of spondylolisthesis and angulatory changes due to the translational force of this position. Furthermore, the bone morphology may evaluate the facet joints, spondylolisthesis, shape of the endplate and Schmorl's nodes. If necessary, the amount of spondylolisthesis should be measured, and it should be obtained or measured down the midline.

In various embodiments, an exemplary method to restore spinal alignment may further comprise an operative protocol or intraoperative method. One exemplary protocol may comprise the steps of: selecting a surgical approach; positioning the patient properly on a surgical table; confirming alignment using one or more intraoperative images; accessing the localized spinal segment in a spine region; selecting proper implant size on at least one side; preparing an intervertebral space within the localized spinal segment (prepare endplate upper vertebral body, prepare endplate of lower vertebral body, pedicle osteotomy and keel cuts/channels) on the at least one side; and/or deploying or implanting the fusion device on the at least one side. Similar steps may be repeated for the opposing side of the vertebrae and/or other treated spinal levels as desired.

If desired, one or more fusion implant components may be deployed into multiple intervertebral segments. The one or more implants are deployed at an orientation within two or more intervertebral spaces in one or more spine segments of one or more spine regions. Accordingly, a first one or more implants are deployed at a first orientation into first side of a first intervertebral space within a first spinal segment, and a second one or more implants are deployed at a second orientation into a second side of a second intervertebral space within a second spinal segment. The first side and the second side may comprise the same side or different sides. The sides may be right side or left side. The first spinal segment and the second spinal segment may comprise the same spinal segment or different spinal segments. The first spinal region and the second spinal region may comprise the same region or different regions. The first intervertebral space and the second intervertebral space may comprise the same intervertebral space or different intervertebral spaces. The orientation, a first orientation and/or second orientation may substantially align and/or follow along the transverse pedicle angle of at least one side, a right side and/or a left side. The orientation, a first orientation and/or second orientation may align or follow along the transverse pedicle angle of at least one side, a right side and/or a left side. The one or more implants may comprise the same height or a different height. The one or more implants may comprise the same length or a different length. The one or more implants may comprise the same width or differing widths. The spine region may include cervical, thoracic, lumbar and/or sacral regions. The orientation, the first orientation and/or second orientation may comprise an angle, including, but not limited to, angles of 0 degrees to 60 degrees; and/or 60 degrees or less.

FIGS. 10A through 10C and 11A through 11C depict one or more exemplary fusion implants deployed at varying orientations. In the embodiment of FIGS. 10A through 10C, the implant components can substantially align and/or follow along the transverse pedicle angles α1 and α2, which may be the same or differing angles, depending upon anatomical variability. In the embodiment of FIGS. 11A through 11C, one of more of the implant components may be substantially aligned with an A-P (e.g., anterior-posterior) axis 1100 of the vertebrae, or portions thereof. In still other embodiments, one implant may align with a transverse pedicle angle and the other implant may align with the A-P axis, and so on. Each of the implant orientations, such as the first orientation and/or the second orientation, may be the same or different within a single spinal level or within or between multiple spinal segments. In a similar manner, the relative alignments of the lower surfaces of the right and left implants may be the same or may differ in location and/or angulation, as well as the related resection depths and/or planar angulations on the right and left sides of the lower vertebral body.

A wide variety of surgical access procedures and techniques may be utilized to implant the devices described herein. Such surgical approaches may include, but are not limited to, standard surgical fusion approaches, including posterior lumbar interbody fusion (PLIF), transforaminal lumbar interbody fusion (TLIF), minimally invasive transforaminal lumbar interbody fusion (MI-TLIF), oblique lumbar interbody fusion/anterior to psoas (OLIF/ATP), lateral lumbar interbody fusion (LLIF), anterior lumbar interbody fusion (ALIF) and/or any combinations thereof. In one preferred embodiment, devices may be implanted into a patient using a posterior transforaminal approach similar to the known TLIF (transforaminal lumbar interbody fusion) or PLIF (posterior lumbar interbody fusion) procedures. PLIF style approaches are generally more medial and rely on more retraction of the traversing root and dura to access the vertebral disc space. The space between these structures is known as Kambin's triangle. TLIF approaches are typically more oblique, requiring less retraction of the exiting root, and less epidural bleeding with less retraction of the traversing structures. It is also possible to access the intervertebral space using a far lateral approach, above the position of the exiting nerve root and outside of Kambin's triangle. In some instances, it may be possible to access the intervertebral space via the far lateral without resecting the facets. Furthermore, a direct lateral approach through the psoas is known. This approach avoids the posterior neural elements completely. Embodiments disclosed herein may adopt any of these common approaches, combinations thereof and/or other know approaches and/or surgical access techniques, including minimally invasive techniques and/or the use of visualization and/or haptic/robotic surgical equipment and techniques.

In various embodiments, some or all of the affected disc and surrounding tissue may be removed via the foramina. The superior endplate of the vertebra may be milled, rasped, or otherwise resected to match the profile of the outer contact surface of the lower joint component to normalize stress distributions on the endplate, and/or to provide initial fixation prior to bone ingrowth. The preparation of the endplate of vertebra may result in a flattened surface or in surface contours such as pockets, grooves, or other contours that may match corresponding features on the outer contact surface. The inferior endplate of the vertebra may be similarly prepared to receive the upper joint component to the extent allowed by the exiting nerve root and the dorsal root ganglia. In various embodiments, the natural facet joint and the corresponding articular processes can be rasped and/or prepared to accommodate and/or support an outer surface of the bridge component.

One exemplary intraoperative procedure or intraoperative method can comprise the step of preparing the intervertebral space within a localized spinal segment for realignment and/or fusion. The step of preparing the intervertebral space within a localized spinal segment on at least one side can comprise one or more of the steps of: preparing the caudal vertebral body on the at least one side; preparing the cranial vertebral body on at least one side; completing at least one caudal keel channel on the caudal vertebral body on at least one side; and completing at least one cranial keel channel on the cranial vertebral body on the at least one side and verifying alignment.

At various points during the surgical procedure (e.g., prior to, concurrent with and/or after the caudal vertebral body is prepared), the surgeon may elect to remove and/or resect a portion of at least one pedicle on at least one side of the caudal vertebral body to create a resected pedicle surface. The resected pedicle surface may comprise a flat, planar plane or surface and/or parallel to the native pedicle surface. The resected pedicle surface may comprise an angle including, but not limited to, an angle of 0 degrees to 40 degrees or more. The angles may comprise a sagittal angle, a coronal angle, a transverse pedicle angle (e.g., a convergence angle) and/or any combinations thereof. The angle of the resected pedicle surface can be angled relative to the native pedicle surface or plane. The resected pedicle surface may be below and parallel relative the native pedicle surface. The resected pedicle surface may be below and angled relative to the native pedicle surface. The resected pedicle surface may comprise a flat and angled surface. The resected pedicle surface may comprise a resected shape, a second resected length, a second resected width of the spinal implant. Furthermore, the angle of the resected pedicle surface may match or substantially match the angle of correction of the optimal sagittal and/or coronal resection plane obtained from the preoperative procedure.

The first resected surface may be continuous with the resected pedicle surface. Alternatively, the first resected surface may not be continuous with the resected pedicle surface. A first angle of the first resected surface may comprise the same angle as a second angle of the resected pedicle surface. Alternatively, the first angle of the first resected surface may comprise a different angle as a second angle of the resected pedicle surface. Also, each of the first resected shape, the first resected length, the first resected width and/or the first resected depth may comprise the same as each of the second resected shape, the second resected length, the second resected width, and/or the second resected depth. Alternatively, each of the first resected shape, the first resected length, the first resected width and/or the first resected depth may comprise different dimensions as compared to each of the second resected shape, the second resected length, the second resected width, and/or the second resected depth.

In some embodiments, the step of preparing at least a portion of the superior endplate of a caudal vertebral body and the removing or resecting a portion of at least one pedicle on at least one side may comprise a continuous, single resected surface. If desired, a surgeon can prepare at least a portion of the superior endplate and the pedicle on the caudal vertebral body by utilizing a powered tool having a flat reciprocating rasp operating at a desired speed until the posterior aspect is flush with the vertebral body and a slight bleeding is observed to facilitate bony ingrowth to the spinal implant after deployment to create a resected surface. Alternatively, the surgeon may prepare at least a portion of the superior endplate and the pedicle on the caudal vertebral body by following along, matching or substantially matching the pedicle transverse angle (e.g., the pedicle axis) while utilizing the long, flat rasp as a desired speed until the posterior aspect is flush with the vertebral body and a slight bleeding is observed to facilitate bony ingrowth to the spinal implant after deployment to create a resected surface. The transverse pedicle angle may include 0 degrees to 45 degrees.

Once the desired anatomy has been prepared, the surgeon may deploy at least one fusion implant into the at least one side, including aligning each of the superior and/or inferior keels on the spinal implant to each of the caudal and/or cranial keel channels on at least one side; securing the fixation screws into the cephalad and/or caudal vertebral bodies on the at least one side. If desired, similar steps may be taken on the opposing side of the vertebrae.

FIGS. 12A and 12B depict one additional exemplary embodiment of an interbody fusion device constructed in accordance with the teachings of the present invention. While this embodiment incorporates many similar components as those previously described, this embodiment utilizes a stepped cephalad keel 55a to desirably secure the device to the cephalad vertebral body rather than a cephalad fixation screw and associated components previously described. In addition, the body 15a can incorporate a block 16a of material that can desirably accommodate and/or promote fusion between the adjacent vertebrae, such as a graft block or similar substances, if desired.

FIGS. 13A and 13B depict another exemplary embodiment of an interbody fusion device constructed in accordance with the teachings of the present invention. While this embodiment incorporates many similar components as those previously described, this embodiment incorporates a body 15b having a mesh, bag or flexible region 16b which may be filled or packed with bone grant or similar material to desirably promote arthrodesis.

FIG. 14 depicts another exemplary embodiment of an interbody fusion device constructed in accordance with the teachings of the present invention. FIGS. 15A through 15J depict various view of still another exemplary embodiment of an interbody fusion device constructed in accordance with the teachings of the present invention, wherein the body of the implant includes various open graft window and/or side ports for graft cell placement, ingrowth and/or on-growth. As best seen in FIG. 15H, a series of teeth openings or pores 1500 can be incorporated into a bone facing surface of the implant. FIGS. 16A through 16J depict various view of another exemplary embodiment of an interbody fusion device constructed in accordance with the teachings of the present invention, wherein the body of the implant includes various open graft window side ports and/or other openings 1610 for graft cell placement, ingrowth and/or on-growth.

FIGS. 17A through 17C depict various view of another exemplary embodiment of an interbody fusion device 1700 constructed in accordance with the teachings of the present invention, wherein the body 1710 includes retractable/deployable anchors or spikes 1720 (e.g., retractable “keels”) which can extend outward of the device body 1710. In this embodiment, the movable spikes can desirably act as keels upon initial insertion of the implant into the intervertebral space (if the tip of one or more spikes is deployed at least partially outside of the device body, such as shown in FIG. 17B), but which spikes can then be used to fixate the implant within the superior and inferior vertebral bodies when fully extended.

FIGS. 18A through 18H depict various view of another exemplary embodiment of an interbody fusion device constructed in accordance with the teachings of the present invention, which includes a plurality of cephalad fixation bores 1810 to desirably accommodate one or a plurality of fixation screws (not shown). Desirably, this embodiment will allow a surgeon to select a preferred trajectory for one or more fixation screws into the cephalad vertebral body, as well as allow the use of two or more “splayed apart” fixation screw to secure the implant to the vertebral body at the surgeon's option.

FIGS. 19A through 19I depict various view of another exemplary embodiment of an interbody fusion device constructed in accordance with the teachings of the present invention, which includes an upper tab 1910 having a tab opening 1920 to accommodate an upper fixation screw (not shown) for securement through a posterior cortical rim wall of an upper vertebrae. As best seen in FIGS. 19H and 19H, graft chambers 1930 are provided to accommodate bone graft or similar materials to desirably promote fusion.

FIGS. 20A through 20I depict various view of another exemplary embodiment of an interbody fusion device constructed in accordance with the teachings of the present invention, which includes an upper tab 2010 as well as a lower tab 2020, the upper tab including an upper bore 2015 to accommodate a fixation screw (not shown) for securement to an upper vertebrae, the lower tab including a lower bore 2025 to accommodate a fixation screw (not shown) for securement to a lower vertebrae. In addition, the one or more keels 2030 in this embodiment may optionally comprise an undulating or serrated keel design.

In the various embodiments disclosed herein, a spinal fusion implant system will desirably comprise an insert body and at least one fixation screw. The fusion spinal implant can further comprise an insert body, a bridge and at least one fixation screw. Alternatively, the fusion spinal implant further comprises an insert body, a bridge, a first fixation screw and a second fixation screw. The fusion spinal implant further comprises an insert body, a first fixation screw and a second fixation screw. The insert body may comprise a single-piece unit or may be formed in a multi-piece unit. The fusion spinal implant may further comprise one or more bone fillers and/or openings/voids to accommodate the same.

In various embodiments, the insert body typically can comprise a top surface, a bottom surface, an anterior surface, a posterior surface, a first side surface and a second side surface. If desired, the top surface, the bottom surface, the anterior surface, the posterior surface, the first side surface and/or a second side surface may comprise flat or planar surfaces. In other embodiments, at least a portion of the top surface, the bottom surface, the anterior surface, the posterior surface, the first side surface and/or a second side surface may comprise a flat or planar surface. In still other embodiments, the top surface, the bottom surface, the anterior surface, the posterior surface, the first side surface and/or a second side surface may comprise curved surfaces. In another embodiment, at least a portion of the top surface, the bottom surface, the anterior surface, the posterior surface, the first side surface and/or a second side surface comprises a curved surface.

Accordingly, at least a portion of the top surface, the bottom surface, the anterior surface, the posterior surface, the first side surface and a second side surface of the insert body may comprise a coating and/or a surface texture to desirably help facilitate healing, osseointegration, and/or to better accommodate loading forces to decrease wear. At least one or more of at least a portion of the top surface, the bottom surface, the anterior surface, the posterior surface, the first side surface and a second side surface of the insert body comprises a coating and/or a surface texture. Alternatively, each of the at least a portion of the top surface, the bottom surface, the anterior surface, the posterior surface, the first side surface and a second side surface of the insert body comprises a coating and/or a surface texture.

In various embodiments, a coating may be disposed onto at least one or more of the top surface, the bottom surface, the anterior surface, the posterior surface, the first side surface and/or a second side surface. For example, at least one or more of the top surface, the bottom surface, the anterior surface, the posterior surface, the first side surface and/or a second side surface comprises the same coating or different coating. Alternatively, each of the one or more of the top surface, the bottom surface, the anterior surface, the posterior surface, the first side surface and/or a second side surface comprises the same coating or different coating.

Exemplary coatings may include inorganic coatings or organic coatings. The coatings may further include a metal coating, a polymer coating, a composite coating (ceramic-ceramic, polymer-ceramic, metal-ceramic, metal-metal, polymer-metal, etc.), a ceramic coating, a bioactive coating, an anti-microbial coating, a growth factor coating, a protein coating, a peptide coating, an anti-coagulant coating, an antioxidant coating and/or any combination thereof. The antioxidant coatings may comprise naturally occurring or synthetic compounds. The natural occurring compounds comprises Vitamin E and Vitamin C (tocotrienols and tocopherols, in general), phenolic compounds and carotenoids. Synthetic antioxidant compounds include a-lipoic acid, N-acetyl cysteine, melatonin, gallic acid, captopril, taurine, catechin, and quercetin, and/or any combination thereof. The coatings can be impregnated, applied and/or deposited using a variety of coating techniques. These techniques include sintered coating, electrophoretic coating, electrochemical, plasma spray, laser deposition, flame spray, biomimetic deposition and wet methods such as sol-gel-based spin-and-dip or spray-coating deposition have been used most often for coating implants.

Exemplary metal coatings may comprise titanium, titanium alloys, cobalt-chrome alloys, platinum and stainless steel, and/or any combination thereof. More specifically, the metal coating includes titanium and/or cobalt-chrome molybdenum (CoCrMo). The polymer coatings may include thermoplastic or thermoset polymers. The polymers may further include carbon fiber, polyether ether ketone (PEEK), polyethylene (PE), ultra-high molecular weight polyethylene (UHMWPE), polycarbonate (PC), polypropylene (PP) and/or any combination thereof. The ceramic coatings may include alumina ceramics, Zirconia (ZrO2) ceramics, Calcium phosphate or hydroxyapatite (Ca10(PO46(OH)2) ceramics, titanium dioxide (TiO2), silica (SiO2), Zinc Oxide (ZnO) and/or any combination thereof.

Exemplary surface textures may comprise a roughened surface and/or a plurality of protrusions. The plurality of protrusions may comprise a plurality of threads, flutes, grooves, knurling, and/or teeth that may include various shapes. The various shapes may include tapered, stepped, conical and/or paralleled, flat, pointed, and/or rounded. At least a portion of the plurality of protrusions may further comprise at least one opening or pores. In another embodiment, each of the protrusions may comprise at least one opening or pores. The plurality of protrusions may be disposed along the length of the insert body and/or along at least a portion of the length of the insert body. The plurality of the protrusions may be disposed onto the insert body following along the longitudinal axis of the insert body.

The surface textures may further comprise roughened surfaces or porous surfaces, including turned, blasting, sand blasting, acid etching, chemical etching, dual acid etched, plasma sprayed, anodized surfaces, and/or any combination thereof. The surface textures may further include a polish surface finish or texture. The polished surface may be accomplished different techniques, mechanical polishing, chemical polishing, electrolytic polishing, and/or any combination thereof. Polished surfaces can be measured in “Ra” micrometers (μm) or microinches (pin.). The Ra may comprise a range of 0.025 to 1.60 μm; may comprise a range of 0.025 to 0.30 μm; may comprise a range of 0.025 to 0.20 μm; may comprise a range of 0.025 to 0.10 μm; and/or may comprise a range of 0.05 to 0.20 μm. Accordingly, the Ra may comprise at least 0.05 μm or higher; at least 0.10 μm or higher and/or at least 0.8 μm or higher. Surface structure is often closely related to the friction and wear properties of a surface. A surface with a large Ra value will usually have somewhat higher friction and wear quickly, and a surface with a lower Ra value will have a lower friction and enhanced part performance and/or prevent or reduce unwanted adhesion of molecules or components to surface(s) (e.g., surfaces are smooth, shiny and less porous). A polished surface has many further advantages, including improving cleanability, increases resistance to corrosion, reduces adhesive properties (for cells or other blood components to attach to), increases biocompatibility, increased light reflection for enhanced radiopacity, etc.

In many of the disclosed embodiments, the insert body may further comprise a first keel and a second keel. The first keel can include a first height, a first width and a first length. The upper or first keel is disposed onto the insert body and/or onto at least a top surface of the insert body. The first keel aligns along the longitudinal axis of the insert body. The upper or first keel desirably extends upwardly from the insert body and/or extends upwardly from the top surface of the insert body. The upper or first keel may extend orthogonally or perpendicular to the insert body and/or may extend orthogonal or perpendicular to a top surface of the insert body. At least a portion of at least one surface of the upper or first keel desirably contacts the vertebra. At least a portion of the at least one surface of the upper or first keel desirably contacts the endplate of a vertebra and/or the endplate of the upper vertebra. At least a portion of the at least one surface of the upper or first keel desirably contacts cancellous and/or cortical bone. At least a portion of the upper or first keel may be undulated and/or serrated.

The first length of the first or upper keel may extend between the posterior end or second end towards the first end or anterior end. The first length of the first or upper keel extends between the posterior end or second end towards the first end or anterior end and extends upwardly away from the insert body and/or the top surface of the insert body. The first length of the first or upper keel may match or substantially match a length of the insert body and/or the upper or first keel may match or substantially match the length of a top surface of the insert body. Alternatively, the first length of the first or upper keel may be smaller than the length of the insert body. The first or upper keel comprises a shape. The shape includes a shape substantially similar to a trapezoid, trapezium, rhombus, parallelogram and/or a sloped rectangle. The first end or anterior end of the upper or first keel can optionally be sloped or at an angle to facilitate easier positioning and/or atraumatic insertion. In another embodiment, the first length of the first keel may comprise or function as an additional structural support component to the insert body, including acting similar to structures such as a truss, I-beam or H-beam. Such structural components may be helpful in supporting the insert body to provide a more rigid structure, resist bending and/or resist shear when coupled to the posterior wall or tab.

In various embodiments, the insert body may further comprise a second keel. The second keel can include a second height, a second width and a second length. The lower or second keel is disposed onto the insert body and/or onto at least a bottom surface of the insert body. The second keel aligns along the longitudinal axis of the insert body. The lower or second keel desirably extends downwardly or inferiorly from the insert body and/or extends downwardly or inferiorly from the bottom surface of the insert body. The lower or second keel may extend orthogonally or perpendicular to the insert body and/or may extend orthogonal or perpendicular to the bottom surface of the insert body. At least a portion of at least one surface of the lower or second keel desirably contacts the vertebra. At least a portion of the at least one surface of the lower or second keel desirably contacts the endplate of a vertebra and/or the endplate of the upper vertebra. At least a portion of the at least one surface of the lower or second keel desirably contacts cancellous and/or cortical bone. At least a portion of the lower or second keel may be undulated and/or serrated.

The second length of the lower or second keel may extend between the posterior end or second end and the first end or anterior end. The second length of the second or lower keel extends between the posterior end or second end towards the first end or anterior end, and extends downwardly or inferiorly away from the insert body and/or the bottom surface of the insert body. The second length of the second or lower keel may match or substantially match a length of the insert body and/or the lower or second keel may match or substantially match the length of a bottom surface of the insert body. The second or lower keel comprises a shape. The shape includes a shape substantially similar to a trapezoid, trapezium, rhombus, parallelogram and/or a sloped rectangle. The first end or anterior end of the lower or second keel can optionally be sloped or at an angle to facilitate easier positioning and/or atraumatic insertion. In another embodiment, the second length of the second keel may comprise or function as an additional structural support component to the insert body, including acting similar to structures such as a truss, I-beam or H-beam. Such structural components may be helpful in supporting the insert body to provide a more rigid structure, resist bending and/or resist shear when coupled to the posterior wall or tab.

The first length of the first keel and the second length of the second keel may comprise the same length. The first length of the first keel and the second length of the second keel may comprise a different length. The first width of the first keel and the second width of the second keel may comprise the same width. The first width of the first keel and the second width of the second keel may comprise a different width. The first height of the first keel and the second height of the second keel may comprise the same height. The first height of the first keel and the second height of the second keel comprises a different height. The first sloped surface of the first keel and the second sloped surface of the second keel comprises the same slope angle. The first sloped surface of the first keel and the second sloped surface of the second keel comprises a different slope angle. Alternatively, the first length of the first keel may be longer or shorter than the second length of the second keel.

The insert body may further comprise at least one tab. The insert body may further comprise a first tab and a second tab. The least one wall or tab, a first tab and/or second tab can desirably function as a positive stop limiter or provide tactile feedback to surgeons for proper placement of the spinal implant and/or insert body between the upper and lower vertebra and/or within the disc space. Desirably this structure may prevent the spinal implant and/or insert body from migrating anteriorly in an unwanted manner during placement and/or long-term use, as well as provide additional support and/or stability for the implant as it abuts the relatively stronger cortical rim of the upper and/or lower vertebral bodies.

The least one wall or tab, a first tab and/or second tab may be coupled or integrally formed to the upper or first keel and/or the insert body. The least one wall or tab, a first tab and/or second tab is positioned or disposed on the second end or posterior end of the insert body. The least one wall or tab, a first tab and/or second tab extends upwardly or superiorly from the at least a portion of the top surface of the insert body. The least one wall or tab, a first tab and/or second tab extends downwardly or inferiorly from at least a portion of the top surface of the insert body. The posterior end of the upper or first keel intersects with the anterior facing surface of the least one wall or tab, a first tab and/or second tab of the insert body. The posterior end of the upper or first keel intersects orthogonally or perpendicularly with the anterior facing surface of the least one wall or tab, a first tab and/or second tab. At least a portion of the anterior facing surface of the least one wall or tab, a first tab and/or second tab contacts bone and/or at least a portion of the anterior facing surface of the least one wall or tab, a first tab and/or second tab contacts the posterior facing surface of the vertebra and/or the upper vertebra.

The least one wall or tab, a first tab and/or second tab may include an anterior facing surface and a posterior facing surface that is flat or planar. The least one wall or tab, a first tab and/or second tab may include an anterior facing surface and a posterior facing surface that is not flat, not planar or curved. Furthermore, the position of the least one wall or tab, a first tab and/or second tab may be monitored with fluoroscopy or other visualization methods during surgery to determine the progress of the implantation and to confirm positioning-such as by providing confirmation that the least one wall or tab, a first tab and/or second tab contacts and/or is recessed against a posterior wall of the vertebral body or the upper vertebral body.

The least one wall or tab, a first tab and/or second tab may further comprise at least one tab opening or bores. The least one wall or tab, a first tab and/or second tab may further comprise a two or more tab openings or a plurality of tab openings or a plurality of bores. The at least one tab opening or bore and/or a plurality of tab openings or bore may be sized and configured to receive a portion of the at least one fixation screw, a first fixation screw and/or a second fixation screw. The at least one tab opening and/or a plurality of tab openings may comprise an tab opening or bore central axis. The tab opening central axis may be parallel or substantially parallel to the longitudinal axis of the insert body or spinal implant (see FIGS. 20A-20I). The tab opening central axis may be oblique or angled relative to the longitudinal axis of the insert body or spinal implant (see FIGS. 14 and 19A-19I). Alternatively, each of the plurality of tab openings or bores comprises a plurality of tab opening or bore central axis, each of the plurality of tab openings or bores central axis comprises a different orientation or angle relative to the longitudinal axis of the spinal implant and/or insert body (see FIGS. 18A-18H). Each of the plurality of tab openings comprises a plurality of tab opening central axis, each of the plurality of tab openings central axis comprises a same orientation or angle relative to the longitudinal axis of the spinal implant and/or insert body. The at least one tab opening or a plurality of tab openings are threaded. The orientation or angle may comprise 0 degrees to 30 degrees.

In another embodiment, the insert body may further comprise a body height, a body width and a body length. A plurality of different body heights, body widths and/or body lengths to accommodate different intervertebral disc spacings and/or other anatomical variations. The different insert body heights may include a range of 10 mm to 20 mm; the different heights may include a range of 11 mm to 15 mm; and/or the different heights may include a range of 15 mm to 20 mm. The insert body heights ranges may be incremental by 1 mm or by 0.5 mm. The insert body height may change proportionally to the insert body width, if desired. In another embodiment, the insert body height may stay the same or substantially the same compared to the insert body width.

In another embodiment, the insert body length may comprise different insert body lengths to accommodate different vertebral body sizes and/or other anatomical variations. The insert body lengths may comprise generic lengths such as small, medium, large, and/or extra-large. Alternatively, the insert body lengths may be offered in a range of 20 mm to 40 mm; the range of 25 mm to 35 mm; and/or the range of 30 to 40 mm. The insert body lengths ranges may be incremental by 0.5 mm, 1 mm, 1.5 mm, 1.75 mm, 2 mm; the superior element lengths 122 ranges may be incremental by 0.5 mm or greater.

The insert body widths may comprise a width of at least 10 mm or greater; a width of 12 mm or greater; a width of 15 mm or greater. Alternatively, the insert body width may comprise a width of 10 mm to 20 mm; a width of 10 mm to 15 mm; a width of 12 mm to 15 mm; and/or any combination thereof. The insert body widths may be uniform or non-uniform. The non-uniformity of the insert body widths may include at least a portion that is tapered. The tapered portion includes a smaller width than the insert body width. In another embodiment, the insert body comprises a first width and a second width. The second width may be the same width as the first width. The second width may be a different width as the first width. Alternatively, the second width may comprise a larger width or a smaller width compared to the first width.

In another embodiment, the insert body may further comprise one or more body openings and/or body slots. The one or more body slots or body openings may be disposed on at least one or more of the top surface, bottom surface, a first side surface, a second side surface, a posterior surface and/or an anterior surface. The one or more body slots and/or one or more openings may be disposed on the top surface and extend through the bottom surface. The one or more body slots and/or one or more openings may be disposed on a first side surface and extend through the second side surface. The one or more body slots and/or one or more openings may be positioned at and/or adjacent to the anterior end or anterior facing surface. The one or more body slots and/or one or more openings may be spaced apart at a set distance. The one or more body slots and/or one or more openings may be colinear or coaxial.

Alternatively, the insert body may comprise a first one or more body slots and/or one or more openings and a second one or more body slots and/or openings. The first one or more body slots and/or one or more openings may be disposed on the top surface and extend through the bottom surface. The second one or more body slots and/or one or more openings may be disposed on a first side surface and extend through the second side surface. The first one or more body slots and/or one or more openings may be arranged in repeating rows. The second one or more body slots and/or one or more openings may be arranged in repeating rows. Each of the repeating rows may be parallel and/or offset to the adjacent repeating row. The insert body may be solid, hollow and/or perforated.

In another embodiment, the insert body may comprise a multi-piece unit. The insert body may comprise a superior component, an inferior component and a filler component. The filler component can be disposed between the superior component and the inferior component. The filler component may comprise a polymer, an elastic material, a 3D printed porous material, osteoconductive scaffolds, bone grafts, bone graft substitutes, ceramic, ceramic based substitutes, bioactive particles, tissue scaffolds, and/or any combination thereof.

In the various embodiments disclosed herein, the fusion spinal implant components desirably incorporate a posterior bridge. The bridge comprises a first end, a second end, a bridge bottom surface, at least one top surface and a connection or fixation housing. The first end of the bridge is coupled to the insert body and/or coupled to the posterior facing surface of the insert body. The bridge extends outside the intervertebral disc space towards the posterior direction. At least a portion of the bridge extends posteriorly, outside the intervertebral disc space.

The bridge bottom surface can be typically flat or planar, although a variety of other shapes and/or contours can be provided as necessary. At least a portion the bottom surface of the bridge will desirably contact a pedicle or surface portion thereof, the cancellous bone and/or the cortical bone, and/or any combination thereof. The at least one top surface of the bridge comprises a plurality of faceted surfaces. The plurality of faceted surfaces helps accommodate bone surfaces and/or other tissue. The at least one top surface comprises a concave shape to help accommodate bone surfaces and/or other tissue. The bridge and/or the bottom surface of the bridge helps eliminate or decrease implant subsidence and provides further support in the “posterior” column of the spine. At least a portion of the bridge and/or the bottom surface of the bridge may contact the cancellous bone (e.g., the spongy, porous bone), the cortical bone and/or the endplate. At least a portion of the bridge and/or the bottom surface of the bridge may contact a pedicle and/or a resected pedicle. At least a portion of the bottom surface contacts at least a portion of the top surface of the pedicle and/or resected pedicle, at least a portion of the cortical bone and/or at least a portion cancellous bone.

The bridge further comprises a bridge length and a bridge width. The bridge length may comprise at least 15 mm or greater, 20 mm or greater, and/or at least 25 mm or greater. The bridge length may match or substantially match a pedicle length at one segment level and/or at each segment level. Alternatively, the bridge length may be the same at each segment level or different segment levels. Each of the bridge lengths at each of the different segment levels may comprise the same bridge length or it may be different.

The bridge width may comprise at least 5 mm or greater; at least 7 mm or greater; at least 10 mm or greater; and/or at least 10 mm or less. The bridge width may match or substantially match the pedicle width at one or a single segment level at one or both sides (right and left sides). Alternatively, the bridge width may match or substantially match the pedicle width at each different segment level at one or both sides (right and left sides). Each of the bridge widths at each of the different segment levels may comprise the same bridge width and/or a different bridge width. The bridge width is smaller than the insert body width.

In another embodiment, the bridge may comprise a fixation housing. The fixation housing comprises a plurality of top surfaces. The plurality of top surfaces may comprise a flat or planar surface and/or include an angled surface. Accordingly, the plurality of top surfaces may comprise curved or arched in a convex shaped surface. The plurality of top surfaces may comprise a curved or convex shape and an angled orientation or an angle. Furthermore, each of the plurality of top surfaces may comprise the same surface type and/or a different surface type. The surface type may include flat or planar, curved, angle and/or any combination thereof. The angle, the angled surface and/or the angled orientation may comprise a range of 1 degree to 20 degrees; a range of 1 degree to 10 degrees; a range of 1 to 5 degrees; a range of 5 degrees to 10 degrees; a range of 8 to 10 degrees; a range of 10 degrees to 15 degrees; and/or a range of 15 degrees to 20 degrees. Angled surfaces help facilitate atraumatic insertion or deployment between the vertebrae. In one embodiment, the fixation screw housing comprises a first top surface and a second top surface.

The fixation housing can be spaced apart from the bridge to form a recessed retention clip channel or recessed clip channel. Alternatively, the retention clip channel is disposed between at least a portion of the bridge and the fixation screw housing. The retention clip channel is disposed between the posterior end of the bridge and the fixation screw housing. The retention clip channel is sized and configured to receive the retention clip. Alternatively, at least a portion of the retention clip channel is sized and configured to receive the retention clip. The retention clip is inserted into and/or disposed into the retention clip channel until an audible sound is created or a “click” to ensure that the retention clip is secured. At least one surface on the retention clip is flush or substantially flush to the fixation housing and/or a posterior facing surface of the fixation housing.

In various embodiments, at least a portion of the bridge and/or the fixation housing further may comprise a bore and a bore axis. At least a portion of the bore may further comprise a threaded bore. Alternatively, the bore may comprise a first portion and a second portion. The first portion of the bore may match or substantially match the head of a fixation screw. The second portion of the bore may match or substantially match the shaft and/or threads of the fixation screw. The second portion of the bore may comprise threads. In another embodiment, the first portion comprises a first diameter and the second portion comprises a second diameter. The first diameter is larger than the first diameter.

The bore and/or the bore axis may be positioned at an angle and/or at an oblique angle. The angle may comprise at least 20 degrees from the bore central axis or greater. The angle may be within a range of 15 degrees to 25 degrees; within a range of 15 degrees to 20 degrees; within a range of 20 degrees to 25 degrees. In another embodiment, the angle of the bore and/or the bore axis may match or substantially match the sagittal pedicle angle at one segment level. Alternatively, the angle of the bore and/or the bore axis may match or substantially match the sagittal pedicle angle at a plurality or different segment levels. Accordingly, the angle of the bore and/or the bore axis may be different at a plurality of spine segment levels.

Accordingly, at least a portion of the bridge and/or a portion of the top surface, the bottom surface, or side surfaces of the bridge may comprise a coating and/or a surface texture to desirably help facilitate healing, osseointegration, and/or to better accommodate loading forces to decrease wear. At least one or more of at least a portion of the top surface, the bottom surface, the side surfaces of the bridge comprises a coating and/or a surface texture. Alternatively, each of the at least a portion of the top surface, the bottom surface, the side surfaces of the bridge comprises a coating and/or a surface texture.

With reference to FIGS. 21A through 21E and 22A through 22E, the spinal implant system may further comprise at least one fixation screw and/or a plurality of fixation screws. Also, the spinal implant system may further include a first fixation screw and a second fixation screw. The at least one fixation screw desirably comprises a head, a shaft, threads, a tip and a screw total length. The shaft comprises a minor diameter or shaft diameter. The threads comprise a major diameter or thread diameter and a pitch. At least a portion of the screw is designed and configured to be disposed into the bore of the inferior element. Alternatively, the screw is designed and configured to be disposed into the bore. The fixation screw shaft and threads are solid. Alternatively, the fixation screw shaft and threads may be hollow to allow guidewires or cannulas through the cannula opening (not shown).

In one embodiment, the head is sized and configured to fit or be disposed within the first portion of the bore of the fixation housing disposed on the bridge. The shaft and the threads are sized and configured to be disposed within the second portion of the bore. The head comprises a top surface and a driving style or drive recess. The head comprises at least one selected from a hex head, a pan head, a flat head, a round head, an oval head, a truss head, a socket head, a button head, a fillister head, an indented head, and/or any combination thereof. The drive recess may comprise a Phillips, a Frearson, a Posidrive, a Slotted, a Combo, a Hex Socket, a Square, a Torx, a Supadriv, a Spanner, hexalobular and/or any combination thereof. The drive recess extends from the top surface towards the shaft. The drive recess is sized and configured to receive a driving tool (not shown).

At least a portion of the top surface of the head contacts a portion of the retainer clip. At least a portion of the top surface contacts a portion of the flanges of the retainer clip. Alternatively, at least a portion of the top surface contacts a flange surface of the flanges of the retainer clip. Furthermore, at least a portion of the top surface of the head sits or positioned equal to the contact surface of the bore disposed within the fixation housing of the bridge. At least a portion of the top surface sits or is positioned below the contact surface of the opening of the fixation housing of the bridge.

The at least one fixation screw and/or one or more fixation screws comprises a total screw length. The total screw length may match or substantially match pedicle length. The total screw length may comprise a range of 30 mm to 60 mm; a range of 30 mm to 50 mm; a range of 30 mm to 40 mm; a range of 35 mm to 45 mm; and/or any combination thereof. The total screw length 248a, 248b may be sufficient to engage with cortical bone, cancellous bone, and/or cortical and cancellous bone.

The at least one fixation screw and/or one or more fixation screws further comprises threads. The threads may comprise a single lead or multiple lead or multi-start threads. In one embodiment, the threads may comprise a double-lead, a triple-lead and/or a quad-lead threads. The threads may further comprise a pitch. The pitch may comprise a fine or coarse pitch. In one embodiment, the pitch comprises a coarse pitch. The coarse pitch is designed to anchor into the softer, spongy bone. The fine pitch is designed cortical bone because the bone is denser, and the torque may be high. In one embodiment, the pitch may comprise a range of 2 mm to 5 mm; may comprise a range of 3 mm to 5 mm; may comprise a range of 3 mm to 4 mm; and/or may comprise a range of 3 mm to 3.5 mm. Alternatively, the pitch may comprise at least 2.5 mm or greater; may comprise at least 3.0 mm or greater; may comprise at least 3.20 mm or greater; it may comprise at least 3.5 mm or greater; it may comprise at least 4 mm or greater. The threads may comprise a clockwise or counterclockwise rotation.

In one embodiment, the threads may comprise a thread diameter or major diameter. The thread diameter may comprise a small, medium or large diameter. Large diameter threads and higher or coarser pitch offer a greater surface area for the purchase of the threads on the cancellous bone. Furthermore, large diameter threads increase the pull-out strength or pull-out resistance-the large diameter threads form companion threads in the bone by compression as well as by deforming the bone trabeculae. The spring or elastic reaction occurs as the cancellous bone is deformed during the thread forming procedure resulting in the compressed companion threads of the cancellous bone to contact the larger surface area of the threads. Alternatively, smaller diameter threads and finer or lower pitch also increases holding power or pull-out strength of the fixation screw. More turns may be completed to engage to a given depth into the bone—the more threads engage, the greater the pull-out resistance. The smaller diameter threads cut into bone while it is inserted cause an elastic reaction of the bone to grip the bone surfaces together—causing elastic deformation of the bone. The bone deforms and offers an elastic binding force.

In another embodiment, the thread diameter may comprise a range of 3 mm to 10 mm; may comprise a range of 3 mm to 8 mm; may comprise a range of 3 mm to 6 mm; and/or may comprise a range of 4 mm to 5 mm. Accordingly, the thread diameter may comprise at least 3.5 mm or greater; may comprise at least 4 mm or greater; may comprise at least 4.5 mm or greater; and/or may comprise at least 5 mm or greater. Alternatively, the thread diameter and/or the threads may match or substantially match the pedicle width.

In another embodiment, the threads of the fixation screw may comprise different thread forms. The thread forms may comprise V-thread, buttress, unified, metric, square, ACME, helical and/or any combination thereof. The helical threads allow the user to transform smaller radial movement into large axial movement. In one embodiment, the thread form of the threads comprises a helical thread form. In another embodiment, the fixation screw may comprise different screw tips or points to properly cut and affix to different bone types. More specifically, the threads may include threads known in the art that can properly cut and affix to cancellous and/or cortical bone.

In another embodiment, the threads may comprise one or more thread angles. Each of the one or more thread angles may comprise the same angles. Each of the one or more thread angles may comprise different angles. Alternatively, the threads may comprise a first thread angle and a second thread angle. The first thread angle and the second thread angle may comprise the same angle. The first thread angle and the second thread angle may comprise a different angle. The thread angles may comprise a range of 60 degrees to 120 degrees; may comprise a range of 70 degrees to 120 degrees; may comprise a range of 80 degrees to 120 degrees; and/or may comprise a range of 85 degrees to 120 degrees. Accordingly, the first thread angle may comprise at least 75 degrees or greater; may comprise at least 80 degrees or greater; and/or may comprise at least 85 degrees or greater. The second thread angle may comprise at least 105 degrees or greater; it may comprise at least 110 degrees or greater; and/or it may comprise at least 115 degrees or greater.

In another embodiment, the shaft of the at least one fixation screw and/or one or more fixation screws comprises a minor diameter. The minor diameter may be uniform or non-uniform. The minor diameter may be tapered. The minor diameter may be tapered along the screw length. The minor diameter may be tapered along a portion of the screw length. Alternatively, at least a portion of the minor diameter may be tapered along a portion of the screw length. The tapering comprises a taper angle, the taper angle may comprise at least 5 degrees to 10 degrees; may comprise at least 7 degrees to 10 degrees; may comprise at least 8 degrees to 10 degrees; and/or may comprise at least 8 degrees to 9 degrees. Accordingly, the taper angle may be at least 5 degrees or greater; the taper angle may be at least 7 degrees or greater; the taper angle may be at least 8 degrees or greater; the taper angle may be at least 8.5 degrees or greater; and/or the taper angle may be at least 10 degrees or greater or 10 degrees or less. In another embodiment, the minor diameter may comprise a diameter of 1.5 mm or greater; it may comprise a diameter of 2.0 mm or greater; it may comprise a diameter of 2.25 mm or greater; and/or it may comprise a diameter of 2.5 mm or greater and/or 2.5 mm or less.

In another embodiment, the at least one fixation screw and/or one or more fixation screws comprises a tip. The screw points or tips may comprise self-drilling, self-piercing, self-tapping, and/or a combination thereof. The long, sharp screw points or tips would desirably help eliminate hole preparation (no punching, pre-drilling or tapping required) and/or help penetrate the bone quicker or quickly and/or capture bone chips or bone debris for increasing local bone density and/or increase the bone's ability to withstand “back out” pressure (e.g., less loosening or migration).

With reference to FIGS. 23A through 23D and 24A through 24C, the spinal implant system can further include one or more retainer clips. The one or more retainer clips may be disposed into to a recessed clip channel that is positioned between the fixation housing and the posterior end of the bridge to help prevent migration of the fixation screw after deployment and/or securement to the bone. The one or more retainer clips may be disposed onto a portion of the insert body and/or onto a portion of the posterior surface of the insert body. At least a portion of the one or more retainer clips is movable from a first position to a second position, the first position being moved axially away from the central axis while the one or more fixation screws are being secured to the bone, and the second position that allows the retainer clip to return to rest once the head of the fixation screw is below the at least one flange.

The one or more retainer clips comprises a body and at least one flange. The body of the retainer clip comprises a shape, the shape includes a “U” shape. Alternatively, the body comprises a first arm and a second arm. The body comprises a first end and a second end. The at least one flange are disposed at the second end of the body of the retainer clip. The at least one flange extends away from the second end of the body of the retainer clip. Alternatively, the at least one flange extends inwardly towards a central axis of the retainer clip. The at least one flange extends perpendicularly from the second end of the body of the retainer clip. The at least one flange extends from the second end of the body perpendicularly toward the central axis.

In another embodiment, the one or more retainer clips comprises a body, a first flange and a second flange. The body comprises a first arm and a second arm. The body and/or each of the first arm and second arm of the body comprises a first end and a second end. The first flange is disposed at the second end of the first arm of the body. The second flange is disposed at the second end of the second arm of the body. The first flange extends away from the second end the first arm of the body of the retainer clip. The second flange extends away from the second end the second arm of the body of the retainer clip. Alternatively, the first flange and the second flange extends inwardly towards the central axis of the retainer clip. The first flange extends perpendicularly from the second end of the first arm of the body of the retainer clip. The second flange extends perpendicularly from the second end of the second arm of the body of the retainer clip. The first flange extends from the second end of the first arm of the body perpendicularly toward the central axis.

The body is desirably sized and configured to be disposed and/or positioned into the recessed clip channel. The body comprises a clip width, the clip width may match or substantially match a width of the recessed clip channel. The at least one flange the first flange, and/or the second flange extend into the opening of the inferior element. Alternatively, at least a portion of the at least one flange extends into a portion of the opening of the inferior element. The at least one flange comprises a flange surface. Alternatively, the first flange comprises a first flange surface. The second flange comprises a second flange surface. The flange surface, the first flange surface and/or the second flange surface faces towards the top head surface of the fixation screw. At least a portion of the top head surface contacts a portion of the at least one flange surface, the first flange surface, and/or the second flange surface of the flanges of the retainer clip. The at least one flange, the first flange and/or the second flange further comprises rounded or radiused edges to facilitate easier insertion of the one or more fixation screws. Accordingly, at least a portion of the body may comprise filleted or beveled edges and/or at least a portion of the body may comprise filleted or beveled edges surrounding the perimeter.

In another embodiment, the insert body comprises an insert body material, the bridge further comprises a bridge material, the one or more fixation screws comprises a fixation screw material, and/or the one or more retainer clips comprises a retainer clip material. The materials may include metal, polymers and/or ceramics. The metals may comprise titanium, titanium alloys, cobalt-chrome alloys, platinum, stainless steel and/or any combination thereof. More specifically, the metal may include titanium and/or cobalt-chrome molybdenum (CoCrMo). The polymers may include thermoplastic or thermoset polymers. The polymers may further include carbon fiber, polyether ether ketone (PEEK), polyethylene (PE), ultra-high molecular weight polyethylene (UHMWPE), polycarbonate (PC), polypropylene (PP) and/or any combination thereof. The ceramics may include alumina ceramics, Zirconia (ZrO2) ceramics, Calcium phosphate or hydroxyapatite (Ca10(PO46(OH)2) ceramics, titanium dioxide (TiO2), silica (SiO2), Zinc Oxide (ZnO) and/or any combination thereof. The materials may be manufactured using traditional methods and/or using 3D printed techniques known in the art. Furthermore, the material may comprise a porous material, the porous material includes porous metal, porous polymer, porous ceramic and/or any combination thereof.

In another embodiment, the polymer materials may be further antioxidant stabilized. The stabilized antioxidants may comprise Vitamin E or Vitamin C. The antioxidants may be incorporated, diffused or doped into the material by blending the antioxidant into the material for subsequent cross-linking and/or diffusing the antioxidant into the material. The material may be further cross-linked before or after stabilizing with an antioxidant. The insert body material may comprise the same or different material as the bridge material.

In another embodiment, the fusion spinal implant system may further comprise and/or incorporate one or more bone fillers. The one or more bone fillers may include hydroxyapatite, collagen, bone cement, allograft, autograft, bone graft and/or synthetic bone substitutes. The synthetic bone substitutes may comprise calcium sulfate, calcium phosphate cements, beta-tri- calcium phosphate ceramics, biphasic calcium phosphates, bioactive glasses, and/or polymer-based bone substitutes.

### INCORPORATION BY REFERENCE

The entire disclosure of each of the publications, patent documents, and other references referred to herein is incorporated herein by reference in its entirety for all purposes to the same extent as if each individual source were individually denoted as being incorporated by reference.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus intended to include all changes that come within the meaning and range of equivalency of the descriptions provided herein.

Many of the aspects and advantages of the present invention may be more clearly understood and appreciated by reference to the accompanying drawings. The accompanying drawings are incorporated herein and form a part of the specification, illustrating embodiments of the present invention and together with the description, disclose the principles of the invention.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the disclosure herein. What have been described above are examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications and variations that fall within the spirit and scope of the appended claims.

I/We claim:

1. A fusion implant system comprising:
an upper or superior component, the superior component comprising a superior keel, a superior contact surface, and a tab;
a lower or inferior component, the inferior component comprising an inferior keel, and an inferior contact surface;
a bridge or tail, the bridge comprising a bridge contact surface and a fixation housing, the bridge extending from a posterior end of the inferior component, the fixation housing disposed at a posterior end of the bridge, the fixation housing including a bore at a bore orientation, the bore sized and configured to receive at least a portion of a fixation screw; and
a bone filler material, the bone filler material disposed between the superior component and the inferior component.

2. The fusion implant system of claim 1, wherein the superior contact surface of the superior component and/or the top surface of the insert body comprises a coating or a surface texture.

3. The fusion implant system of claim 1, wherein the inferior contact surface of the inferior component and/or the bottom surface of the insert body comprises a coating or a surface texture.

4. The fusion implant system of claim 1, wherein the inferior contact surface of the inferior component, the superior contact surface of the superior component, the top surface of the insert body and/or the bottom surface of the insert body comprises a coating or a surface texture.

5. The fusion implant system of claim 2, wherein the coating comprises a polymer, a metal, a ceramic and/or bioactive coating.

6. The fusion implant system of claim 5, wherein the bioactive coating comprises an osteogenic coating.

7. A fusion implant system comprising:
an insert body, the insert body comprising a first keel, a second keel, a tab, a top surface, a bottom surface, an anterior surface, a posterior surface, a first side surface, a second side surface and an insert body material, at least a portion of the tab extending upwardly from a portion of the top surface, the first keel extending superiorly from the top surface of the insert body and the second keel extending inferiorly from the bottom surface of the insert body; and
a bridge or tail, the bridge comprising a bridge material, a bridge contact surface and a fixation housing, the bridge extending from a posterior end of the insert body, the fixation housing disposed at a posterior end of the bridge, the fixation housing including a bore at a bore orientation, the bore sized and configured to receive at least a portion of a fixation screw.

8. A method of treated a diseased or damaged motion segment of a human spine, comprising:
accessing an intervertebral disc space between a first vertebra and a second vertebra via a posterior surgical approach,
removing at least a portion of an intervertebral disc within the intervertebral disc space via at the posterior surgical approaches,
removing at least a first portion of a superior endplate of the first vertebra to create a first resected surface of the superior endplate,
removing at least a second portion of the superior endplate of the first vertebra to create a second resected surface of the superior endplate,
placing a first insert body on the first resected surface, the first insert body comprising:
a first keel, a second keel, a tab, a top surface, a bottom surface, an anterior surface, a posterior surface, a first side surface, a second side surface, an insert body material, and a first bore at a first orientation, at least a portion of the tab extending upwardly from a portion of the top surface, the first keel extending superiorly from the top surface of the first insert body and the second keel extending inferiorly from the bottom surface of the first insert body;
a first bridge or tail, the first bridge comprising a bridge material, a bridge contact surface and a fixation housing, the first bridge extending from a posterior end of the first insert body, the fixation housing disposed at a posterior end of the first bridge, the fixation housing including a second bore at a second bore orientation;
a first fixation screw, the first fixation screw sized and configured to be disposed into the first bore at a first orientation of the insert body; and
a second fixation screw, the second fixation screw sized and configured to be disposed into the second bore at the second orientation of the fixation housing of the first bridge,
wherein the first insert body inhibits relative motion between the first vertebra and the second vertebra.

9. The method of claim 8, further comprising placing a second insert body on the second resected surface,
wherein the first and second insert bodies inhibit relative motion between the first and second vertebrae.

10. The method of claim 8, wherein at least a portion of the insert body material comprises a polymer.

11. The method of claim 9, wherein the first and second insert bodies are formed in substantially the same size and shape.
